SEQUENCE LISTING (16 sequences)

SEQ ID NO: 1 - DNA, 3112 bp, Homo sapiens
  source 1..3112: mol_type = genomic DNA
gaagtggcattaaatgagctctcagaggcctcccagcagtgacccagaatgaggtgagcagcaaattctcccttgggaaaggcagcagaagccagttgggcagtgggtgctgctcccctgaggttggctcacccatcccccagccccccaccccaggaatagctgtgtgaccttcagcaagttactgagttgatctgtgtctcacgtaagagaaccaacttaattatagttccggcacagtcaggttgctgtcagaattaaatgtgtgaatttgtatgaagggatctgaatagagcctgacttatgggaagtgctgtatttcagcgcacactctgaatagcataatcattactaaaagaattttttttttgagacggggtctcactctgtctcccaggctggagtgcggtggtgtgaacacggctcactacagcctcaaccttccaggctcaggtgatcctcccacatcagcctcctaaacagctgggactacaggtgcaggccactgcaccaggctaattttctgtgggttttgttgttgttattgtttgtagagatgggttttgctatgtttcccaggctggtcttgaactcctgggctcaagtgatctgcccgccttggcaggactacaggtgtgagccattgcaccctgcctaccctggcccattgactattatgaattaaaggctattgaaaaacagcaggtgcatcgctggcaaaagtatacttgtgtaacgaacttgcacattctgcgcatgtatttcagaacttaaagtaaaaataaaataataaagtaggccgggcgtggtggctcacgcctgtaatcccagcactttgggaggccgaggcaggtggatcacaaggtcaggagatagagaccagcctggctaacatggtgaaaccctgtctctactaaaaatacaaaatttagccgggtgtggtggcgggcacctgtagtcccagctacttgggaggctgaggcaggagaatggcgtgaacccaggaggcggagcttgcagtgagctaagatcatgccactgcactccagactgggcgacacagcaagactctgtctccaaaaaattaaaaaaaaaataataaagtattcagggataaagagagaaaaataaatttagattgtatacatatgtaaaatatattttaaaaggtaagcttgtcttttttattccttaaaggagcagatattaaaaaaaaaagaatagaataactcatctttttagcctccacatatttacttgcttcttcccaagttacttggtctaattcagtatataagtaactgactctcactgctttttctgtgccatgtaaaacttgcattgtattaatttgtgtgcttttctcctgtggacctgtcttatgtcaacttaattctcaggcccagccaaaaaaaaaatacccttaaagggagcttagcttgcctccatgaccctcaatggtggctgtaatcaaaccacatcttccaggcaaacaataccacatcctatggccttgcaatggcatggcctccctccagcacagcctctctggaggagggggaggcaagggtggtgagatctcaggcctccctcatgctacctcctattagttatatgacccctggtgtcacctctcccaccacactgtaggctccagggagtaggattcacatggggttcatttgtcctccctgccttcagcagactccaggcacaggtgttcccagagtaaagggctcactgtctgcttcctgggtgaatcattctccccgcaaacccatgaggtgagtgcagtcgtggcttcagctcaccgcctgacaggctccaggctgtagcacagggagggccctgtggactctgctgaggaaccatacgagctcccagaggccaagtgcaggacagagaatagagaagcagctgctcagagaactctagccctgtagaggtgcaaatatggctgctattatacagaaggtgaggccaaaaatgctggagacactgctcctgaatctcagttttgccacttgtaaaacagggaaaacaacctactctcatgtccccaggttgttaggaaaatccactacatgaagatgtgcagtctctggctgggagcaggacaccagctgcccaacagcacaggctcagggcatggcactgtctccactgtccccatccccaacctggaagctagagcctcgctgccctgccacttctggatgcaggacagagaaccggaactgggactgtggtctctcagtagaggctgtgaacagccatctgtggccacacggattgagacctttcctgctcactcttcaaactggggcaccggctgggtgcccaacagaaagacaattggaagagtggcaggtaaaccaggctgtggccctgcagtgccatcaccgtcctcatttgaattctggctcgtctgttcctctacggcccctccgcatggcgctccaccccagtcttcagaaatttctgcctcaagctcagggagctggtactgaggtacaggtaggagaaggtgcagcctctgctgagtggaggacatgtgccctggggatccagagtctagagtgactgacaccagccctgcaatttgtcctcctagacctgttaacagagcctgcagaagtttacacagggctgctgaagatttttatcccctcattttacaggaaggagctggggccataccagagtcagagctggaccgggagcacggcagggcccagccatccacagcctccaatttcagtactctctaacaacctgagagcctgagccttcggatcttcttgattcctactttgataaagagagggatggggttggaaggacctggcatagagattggaccagtccggcattgacagagccaggacctggggtccggaactgagctcagctccaggctcatctgaagtcaccctgacagcctgaggatggagaccttatttccattaacaggcatggcggttggcagggactggccccagctgagcacggttttccaagtacacagctgggaattaagtcttctctgccgccctcccctgtagtcttgcctggatccccccagtcatctactcccagtaccaaaaactcc

SEQ ID NO: 2 - DNA, 14466 bp, Homo sapiens
  source 1..14466: mol_type = genomic DNA
gattctgtcgcgaaccctctcacggcagctatgagactgctgcgctccccagacgaggaaacctggtctcccggagccaggtaacccacccgcagcctgcgggagagccgaggtctgcagccgggccgtcgggctgcctcttcccggggacccgggagcggggggcggggggcatggtggcctctcgggctgctccgaacgtcccctccaaacagcgaggggacgcacagccccactcaggagggagccaatgacattgaacctgaacccaagccagccttagactgaactttcaaagtaccattaagcaacgccgtgaggaaatggaacattcgacaaggtattatgtcgcatctctctctctttttttttttttttaggtggagtctcgctctgtcgcccaggctggagtgcggtggcgcgatctcggctcactgcaagctccgcctcccgggttcacgccattctcctgcctcagcctcccgagtagctgggactacaggtgcccgccaccgcgcccggctaattttttgtatttttagtagagacggggtttcaccaagttagccaggatggtgtcgatctcctgacctcgtgatccgcccgcctcggcctcctacagtgctgggattacaagcgtgagccactgcgcctggtcgcacctcttttcaaaagccagtgttatctgggaaagtgcaaggggtggggagtgggaggtgctgttttcaatgaaaagaaatgaaagagacattgtgcgtcttcttaggtgatattacagtaccacagacactacagttaggtgactgagtccagtattgtctgttttctcaggtgtgatgactgtgtcgtgtcatgcagaaaaatgtccttatttctaggaatcgcatgctgagatctcggggagtaaagggccaggatgtctggaacttaatttaaaatgatttgacaaggacaggcatacacatagggacaaagcaaatatggtagatgttatcaattgctgaatctatgtgctgagtacacacgtatttgttgtattgtcttttcacctttcctatatgtttaaaatctttcataatgtcattcggggggaaaaaaagctgatctggctatacttatttgggaatgtgccggctaaaagggaactaacaaataaaaacttaacttagaacataaaattttagagactgaaggaaccggagaccttctagagctcaactctcattttaaagatgaggaaagtgggcatattgattttatcttattttctttttctctttactttttaaatgtttttggtttaactttaagttcagcggtccatgtgcaggtttgttacacaggtaaacttgtgtcatgggagtttgttatacacattagttcgtcacccagatattaagcctagtacccaatagttgtttttcctgatcctctccctcatcccaccttcaccctccaatagaccccagtgtgtattgttcccttttgtgcccgtaagttctcatcggtcagctcccacttataagtgagaacatgtggtatttggttttctgttcctgtgttagtttgctgaggacaatggcctccagctctatattgaatagttaaaaactatttaggccgggtgcagtggctcacacctgtaatcccagcactttggaaggccgaggcaggtggatcactgaggtcaggagtttgagaccagcctggccaacatggcgaaaccccatctctaataaaaatacgaaacaattagccgggcatggtggcagatacctgtaatcccagctactccggaggctgaggcaggagaatcacttgaacccggaagacggaggttgcagtgagctgagatcacaccactgcattccagcctggtgaccgagcgagactccgtcgcaaaaaaacaaaacaaaacaaacaaaaaaaaaactctttaaaataagtgttataaactaacagtttatttatcccaggcacagaaagacaaatactgcatgatctcacccacaggtggaatcttaaaacattgaactcacagaaacagagtagaatggaggttaccaggggctagagtgtggggggttggggtgatgttggtcagagggtacaaaagttcagttagacaggagaaagtttatcttttagtgattttcatgattttgacaaagttgaaacaactgaaacaaagtatctaaatattttaatacttgtaattattttttaattgttgagtgattttaatgagtcaaaaatataaaacaatcatgcataatacatataaataatttgagtgtccaacccatattacagatgaaagtaaagaacaggcagattctgagacttgggacaggcagcatttggcacctgcggaggcccggttctacacgtctgtagtggcagaaaggcaatatatattaaaacctaggaaaacagtcagggcacggtggctcacacctgtaatcccagcactttgggaggccaaggcgggcagatcaccaaaggtcagaagttccagaccagcctggccaacatggcgaaaccctgcctctactaaaagtacaaaaattagccaggtgtggtggctgtaatcccagctactcggtaggctaagacaggagaatcgcttgaacccggcaggcagaagttgcagtgagccgagatcacgccattgcacttcagcctgggtgacagagggggactccaccaaaaaaaaaccaaaaaacaaaaacaaacaaaccttggaaaatgggtgagagaggatggtatgtgatggttttatcaagtacttctactctatttatatcaactactccaagctgtggtaaacacctctgggaagccaggtccagtgcaagtgggccaaagcccgtattgacagttataaaccaggcatctccagatgacagctttatgacccaagcagtgtgctcctcactagactagtgcagtacgtatcagagtccagcagggagtgtgcccacctggaggaccagggtaaaggtgggaggccaggagcagggatctgggagctaacaataactcaaagtacccacgttcatggtttccaccctccccctgagcccccacgtatgccatccccaaccctctcccccatctttcaagtccatgactgccagtcaccaattagacccatcactcaacagccctggttccccaacagatggactcacaagaacaagaaacaatgactcggtcattagaactcttagtttcccacaattcctgtcattgggattaaagttctccgatgttgtccccatcatgtgtctgagaaactggccatgggcacctcctgaaaggggaagcaagagtgactctgggggcttgttgtgctcctcggaagaggttctcactcctttagcatgtcgctgcctggccctggacagcaaaaaccaaccctttgagaggacttaagtaaatacatcatcagatctgggagctgatcaaaaccttatttcgttgcactcacatgtatgtgtttcttaatgtacttgatcttggataaaatatatcctagtgcattagattttaagccatggatacacatcctaagttgaatatccagttatattaatttcattcatgttaattgatataaatattaatttatggtgaattatagatttatttaactgatgtagtagtgatatatacataagaattcctggaaggagatgaattcatataagctgtgtagagaaacctatattctttcaagtcatcacagatactttataaaagtaggtacttcgggtaccaaatactgtagtgtccccagcaaatctgcagcaattggcttttgacactgaaatgttggattccgccctgttgggtagtgttgcttgttcgggctttagagttaagattaaatcgggtggactatgtggatcctggaaactgtgtggggtgttagacttagaggaacgcacagagcttccatggctgaccttgacctcgggaggtggccagggacctggcagtctctcatgcaaggctctgctcagagagctgaacactgaccccactgaacgctgtgttaaggagctagggtccccccctccctgcccccagctccctgctatgagctttcccagttactacatgaatccaattacaggcacccctgttgggtttcctgtgaaataacagatcttactgtaaccaaatattaccatctcggaaaaaccttatgcaactagcgaggactcttcattctcaggcagcagagaaaagcctctttgttttggggctgaacacctggaacatgttatccggggcggcctccccgggcccacagtggagagggggcttctaggacccaatcacacccctgtctgtggaggactggctagcccaaagctgcctctcgctggctctgtttagcctggtttcagcagaccctggctcttgcagtcagccataggcaaatggcagagggcgccccgcatcgatacagaaggactacattggcaaggaccacagaactcccaggcaacctttgataagatgagaagaggaagggctaaggatgcagagagatgggaggcctaaaatctaccctccctgcccccagacactggtgccctcctgaagccatcacagataagaatgctggctgtgtgtgcaggagagcaggtgttgtggcatgggctcaatcaggggacatgtctgtggatgactgcccttggaaaccagtgaaaccctgtgatcccacctttccttcagatcactgactttccagaagaaaacttaatgctgctctttcatccttacgccatggctaatacagcagtcacagaaagggatggctgtaagaacccaacgcaggccaggctagaggcaggagtcaaggccggctgccgtagggctcaggatgtccatccgaccgtgacctcagcactcattgataaaaggagagtaggaccattcccaggtgtggagaaccagggggcatctatgacaaatgcagacccaaacaccggtgtaactttttgaagataactcccatttttactcacgtctcttagtcttaatgaaatacaaagtccttgctggaagggaacagctgtcactcatctctgggcccctcgttgttcgacacctgaatgtaatgtgtagtcttcaaccctccaaccctctccaccaacactcatgggtgtggcagtgccctggggcctaggagaggcagattctaagctgggcatgaggtaaggtcaaagatagcactttcccaggagctccagggagatgctagggctgtagcacctaggctcagttgaaaaggggtggtccaatgacagttatttgtctacacaataggcaatttccaacatccaataaaatgcctttttttcttcttcttcttctttgagacagagtcttgctctgtcgcccaggctggagtgcagtggcgcgatctcggctcactacaacctctgcctcccaggttcaagtgattctcctgcctcagcctcctgagtagctgggattacaggtatctgccaccacgcccagctaattgtttcgtatttttagtagagacagggtttcaccatgttggccaggatggtctccaactcctgacctcaggtgatccacccacctcggcctcccaaagtgctgagattacaggcgtgacccaccacgcctggccaaaatgcccttgtttgactctgggtatctaagacagttataaaaatatagcttccccccaacccaccaaataacaacaaaatacagttgtgtctgtgtgataattcttgtatcattccttccacccttacctgcgtgtgaatattacctgcaagtctcagcaactcatcagccatgtagctcgtgtagcatcttagatgactatttggaaaaataaagccaaatacacacttttgactcaaaggaggaagcacattcctgtcgagctattgttattttaataattggaaataaattttaataaagccagtaacttttgctaaatacacagttgtgctttgacagtgacggaaaaccatttaaagccacgcttgaggctctgtccttggcacaaacacgattgaaagtctacatctgattgacccttttaaaatgccttcctttacttctgaactgttgataaagagctctatttgtttgtagaatttttccttaaaagtggggcaacagaaattatttcctgtccagcgtcctatttcaaggaacctggtgcgtttctactcatcgaagctggtctctctagcaggatgcaatctggctgcttcgtcctgctttaaagcaactttaatttacttgattaaaagtatgcctatgctcagcacgtctcacaaagtcagggagagacattcatgaagagtttccctttgaaataaaatattcagattttgtttcccagtgtttccacgtctatcttctttgctttcttagttttcatgtggtatccgatatcacagaggctggcagcagaaccgcatggacacgcttctcatgggggcccattctctgggcttggaggtgtaacatatattttacaagaacaatgggaaatcatttagggatccacttttcaccattctcttaggagaaatgctctctgggttggaagagggggtggagggcagatgactgttatataaagcgcttgacaggaaaaaaggcaccccctatttcttgggattttctcccactacagtccctttgacaaagttcctttccctagaatgttcccccaaaccgtaaatattagtgaagaaaaagaacttgtattgaccagaactcattacacaatcagaaagattgagcattttatatttgtttttatatttatatctttcgggggaaaatgcactatgtatattaattttaccacaatgtgcagaaattccttaggcaaatatgtaattaggcattttaaaatataaaatgtatttatttatagcttccaaccacacatttatttttatttatctgaaagatttaaacgacactgtcttgaattcgttctttctttagtttatttgcaaaatatataattaggcaaatagccctgaggagactgggcagtgatctttctgcagcttttaaagtattggtaggacgctgttctcttcttttccccaagttcaatagcttagctattcgtttcctgtttctaatagccaaggtaattagaccataatgagaactctttgcttggaagatgacatctgaaagttgaaactatttggctgctcaaacctcaacagggaggggagggggtaagcatcgcctcctcggtgggaggaagttctacgactagagtgtcactttcccctaaacttatttcttcctggaaagactccaaagtccagaaggcaaagtgacatgtgcactggttttttttttttttttttttatggaatggccaattcttcattgttaaggaagcatcgcttaaaaactccaaaaagcccagaacatatagaaaacctgtttgccccttcttttacctttcttttactgtctaaatatcacacacgtacattaaagtgcatacatctgaaaggcatatcttgatgaatgtttcacctgtgtgtatatctgtgtaaccaccaccaatatcaagaaatagaacatttcttgcccctgaaggctctcagacccctccctgtcaagaccccacctgagggagccaccgtgctaatttctatcccccagaatggttttgcctgtccttggattttccataactggaactatgaatgttacatacactctggctggcaggcgctttttctcaagggtgcatgcctgaggtccatctgtgttgctctgtagagttgtgctcttacattttcactgcagttagtgttcctgtgtgtgaatatttatgtatctgttctgccttttacggacattggggtctttcccagttatggctcttaggaataaagctaccatgaacattctcatctacatctctgggtgaaagcactttcttccttggtgaacctaggagtgaacttcccgggccacagaggatgtgcagctttggtagattctcccaaaccatttcccatgagttgtaccaatcatcagtcccaccaggagttcattcaccactttttgatgggcaaccaggggtcaatctcatcattgaaagcaaagtctcaactgttcgcaaaagggggacacgaagacttagtagcaggagagatagaaaaaaaataattaaaagggaaaaagctacccccaagaaaggacctggcccaggctgaacaagctctggcagccctggggcattgctgtggtaaccgtttcccacccttgctgcccaggaggaggggcagtggctctggaggggaagctcagtcccttggggctttccacctcatgcacatggacagacaaggctttcctggggtcaggccacctccagcaccctccagcccagttctcagccacatagagcatcacttgtaggacgagactactggctccagtaagtcccctgaacttttctttaggccaaattccatatgttctctctttccaatactttttccaattattgaccaaaaagtaaatagtaaggctatgtcaggaaatctgctgccttgtggccagcaaccctccccagcccacagtggccccactgcactctgtgtgcctgtcctctgggccagcactgccccggccctccccacttacatgcagccccctgagggtcccagaagggagccttcagtcactgagctctcactaggccagaattccaggaaaggtgagaacccaaaacacctcccctgtctaccccagctgttctagaaatacaggagatggaggttaatagaagaccactgaccttcctgacagcttttctaaacaaccagggcagggtcattttctacatgtggtattctcacggctccctctccaaattgaggatgccccactgacgtctgtgtgggccatcaaagggtgtgtgtgaaaactttcctgcccatcgcaagctagagcctctgggtcaagcccgtggccaggcgagccttgcccatctcctgactccctcactcaatttgccgacttcctgttgggctttctcttagggcctttactctgtcctctctgtctctggctctgatcgtttcaaattgctgccgggagtctcacatataccaggatggatagcagagccttagtcctgaagtttcactcataactcaaagggataggtatattttgttgtagcgcaaaatcgattcccatcccctgggaagcttgtgtgtttagtgtgtacttaggtagtgaggagcccacctgctcactcactttctgcacaaggagcatttctgtctgcccgggcagagacccctgactccaacaggccacacccaccagtggtgaagtgtgctcagccagggaggaagttcttatgggtaggaagagtttcaaatcttccttgacagcccacacagctgtcagagggcatcctcagtttggagagggggccacagaatattaaggtttaaagacgccaaaggctcatgtgcttcccttccttctctctctttgtctctgtttaccatgagactgttttgaaaggtgtgcactctcctcctcttaccctcctggggcttctcctcctgtctcaattggagagggacagggacagatgcagaggcccccggatacaaatgaagagcctgggctgaaccagggccagatgggtctgcagccctgtgatggtcacctggggaactattgtaggtgtggtggttttaagatatgaccccagattctttgactctcctttcttcaagaggtggagcttaatttccctctgcttaaatgtaggctggatttagtgccttgcttctcatgcataaaataaagcagaggtcatgtgtgtgaggtcctaaacagtactgcagtttcctgcttgttctccttctctctctctcatatcgttagctctgggacaaaccagcttctatgtcatgagcacactcaggcagctcattggagagatctgcatggtaaagcaatgaggcctcctgccagcagccatgtgagtggcatcttggagccagaccctccagccccagtcaagccttcggatgcctgcagccctggctgacattttaaccacaacttcatgagagattctgagccagaatcacccagctaagctgctcccgggttgccaggtgagaaaaatgaatggttatgattttaaaccgctaggttttggagtcatttgttacacagcaacagataactaaaacaggaggttattagtactactcaccaaatatttctggttctagcttccagaatcacggtaggatgacctctctcccctgcttggaagttacgtatatccacgcaactttgaaattgagtaagactgggatgtgtcatccccaagagggcacttcaagagccagtgtgccatcacaacctctttctctccctctatcaaaccagttgccagtgttataaataatgagaccatgctctatcagcttggggccaggggttggggtgatgtggagcagagctgttagctgacccaccatggatgtgtagcagcggaagaagcaagccgtgtgctgttgaagcacccacatttttggtgttactgcagcacaacctgaactatactgactgatgcaggtgaggatacacagggatcctctgtccctccacccccagctcttcatgctctttgggcccaggaaagccagtatctccattgaggtcagcaggatcatgcccttgatgtgggttccctgtcccctgcccaggggacctccaccgtgtccacacctggataaaaagtgaggagcttggttcaggagtgggctgggggcctggcctggtcatcacgacctctctgtttagaaccctgtggatgggaaaggcttacatggcagtaagaggagttgaaggtgttaagtaacggcccgtgtgatccagttctgaaaaccacaaagtcaacagtgattgatcctttgctttcttttccagaaccatagagaatttggggaaaatactgtctggccacccagaaaacaaagaccagttaggatttgttttattttctcttcttcccagatcaatccacagttctccagggacccattgtggttttgtgcttcctctgtgcctgttctgctcctctccatgcaccccagcccctcccagtcccctcaacggtccttctggccaggcccatagccccctctctgccccacagtcctcaggctccagctttctcgttaggaagtgttagaaagttgctggagtgagaaatgagcctgtgaaactcgttgtcctaattaaacttccacaaggagagtgacaagaggcgacggggaggaaacggtgccaccaccaaggtctcagcctgggcccaggtcgaggaaggcctttgtcctgcagaacaaaagccactgtttgcacgtgttaaacttcatcaaacttcaacagacccaggtgcaaaaccccacatcccaaatgccatccgggcttatgccaccttcccgagcatggaccgtccctagccgtcaaccctaatcagggccatgccctacatcaaagctgcactccctccacagacagcttctgctctgagatgcattgttcctatgacttcctacactccgcccttgtctgacgaattagggcacggagcccccccatggcacagctgggccctgctggcattgttaatgaggtaggaagggcgcgtttgtctgagcctgggactaacataatcacataaagttgttgctttggaatagatggagctgccccagagtcgattttcttggaagcaacgcaaagcacagcttcctgggagaagacacaatgcctcctattgtctctgccacttcccctccagcctcccagcatttctgcgtctcctcgggagccactgccctgtgcaagagctgtttgctgatgcccagccgcggcttccaaaactcccgcgagaagtcgtttctgtccacagtcattgtttccagaccagccgagccaactggggaggagaggccaagcccagggagagcagtaagaggcccagcttgcaacacaggcccccggcggctggagtcggctcatgggttgctggggggcagctggtgccagccctgctagacatgtccagggtcggggcagaggccagggtggctctcatccacaacgaggctccaggaagaggctccagagggcccaggtccctggaggaagacagagggtggattatccagtgagtcctcagattgctccagggagactctacccctaggccctcatcctacctaggactgaggcagccatctgggtctctgcttccctaaccacttcctcctccacccgaccatcacctagtctcaaggacttgggcagcttctggaagtggctgagactggaagaatgttgtcacacctaccagatatccctacgtgtatctctaccacccccactacatattctgaagaaccaaggtgagctgggggaatccccaggccagcagggcaaggcctgggcaggaatggagtgaggaagtgagaagtggggatcttgggggtgggcgggctgtggggctgggctgtgctgcacactgggcaggaggcaggagatggcaatggaagcgacagctccccatcttccccggctgcatacgtgagggattctgctgcattcactgagtcaccaaaggacactggaaggccatcaggatttttgggtgaccaagacatggctcccctcttactgcctgtggggagacaggcatgtgaacacccaagtctaacccaagctgagttgactgagggccatttaaagattccacagaggctgagagcagtggctcatgcctgtaatcccagcactttgggaggccaaggcaggtggatcacttgaggtcaggagctcgagaccagcctggccaacatggtgaaaccccgtctctactaaaaatacaaaaattagccaggcatggtggtgcacaccagtagtcccagctactcaggaggctgaggcaggagaatcgctggaacccaggaggcaaaggttgcagtgagccacgatcacaccacagtactccagcctaggtgacagagcgagactccgtctcgaaaataaacaaataaagaaaggttccaaggatggtctggaggacaaacaaaggaggagatttggtgcaggagttaggacaaagactcctgggacagcagccctcacttggggccatagtgaatgaatgggggccttttgcagaagggaaatggaggacatgtgtggtgaaggctttgctgaagcatctcttataaaaggggcaaaagcacttgctctcctcccagctctggctcagcctctctctcttcaagtccctggcatccctggaaaaccatgatttttccattctgcagccagcccaggcacctcctcccacccccgcacactgcagtctggtgtgtgtggtgggcacacatagaacacttgctggtcttgaaaatgaagccatcttttggggcccctagaaatcaaagagacccacttagccaagatatcactcttggacaccagcaagggtgagatcacttgataggtattttgccgtgtgtttctttttaactatataaaaaacaaaagggtggatgtgcaaacccaacccaagaataccttggctcaggctcaactagatgaaacttttaaaaatgatttaatgttatgttagactgttgcatattaaagcgatgtaatgccttctaagaaacgagagtcagatcctgaaagcgatgacttgggtaaatgtaaggagttataactcacttttcaagagagcaactataaacattttgcccaatatgtacagacctctaatgacagtgtgcaaagactctttcgttcccagattatgaggctgccactttctcctccccttcctgagccgcacttttctaaacagcaactgtcggggcagggggatgggaagctcatgagagaaggaaatatttgggaccgtgtcggccagtggaatggggcttgtggtcaccgtgttacagagtcaaatcacttcccagtaaacaccagtttccgatggctctctggccagggttggccggggacaagcagggagtatatgtgtcgggtacgttgcgtcacaagctgtgcagctgtgtaggcctctctgtccagaggcattaatggaaaatgattgggcacccagcccacagcagatgcgctgaggaatccagccagggggagctgggatgcagtgggagaggggacacgcagggcctggctgagaagcctcgcgaggtaaccacaggcggaaatctgaaaaatgaagacgttcccaccagttccagaaaacagggctaacttaaaatgcagcccctttccttgaagtgggcttggcaggccccaggtgttgaaaggcaaaagaaaaaaaag

SEQ ID NO: 3 - DNA, 16 bp, synthetic construct
  misc_feature 1..16: note = nucleic acid agent
  source 1..16: mol_type = other DNA
caggagaaaagcacac

SEQ ID NO: 4 - DNA, 16 bp, synthetic construct
  misc_feature 1..16: note = nucleic acid agent
  source 1..16: mol_type = other DNA
attctgggtcactgct

SEQ ID NO: 5 - DNA, 16 bp, synthetic construct
  misc_feature 1..16: note = nucleic acid agent
  source 1..16: mol_type = other DNA
cataatctgggaacga

SEQ ID NO: 6 - DNA, 16 bp, synthetic construct
  misc_feature 1..16: note = nucleic acid agent
  source 1..16: mol_type = other DNA
gtgtggttggaagcta

SEQ ID NO: 7 - DNA, 16 bp, synthetic construct
  misc_feature 1..16: note = nucleic acid agent
  source 1..16: mol_type = other DNA
cagaagcacgagggtt

SEQ ID NO: 8 - DNA, 16 bp, synthetic construct
  misc_feature 1..16: note = nucleic acid agent
  source 1..16: mol_type = other DNA
aagctgaacctgacac

SEQ ID NO: 9 - DNA, 16 bp, synthetic construct
  misc_feature 1..16: note = nucleic acid agent
  source 1..16: mol_type = other DNA
tcgtccagctaataat

SEQ ID NO: 10 - DNA, 16 bp, synthetic construct
  misc_feature 1..16: note = nucleic acid agent
  source 1..16: mol_type = other DNA
ttggacagagtaagca

SEQ ID NO: 11 - DNA, 16 bp, synthetic construct
  misc_feature 1..16: note = nucleic acid agent
  source 1..16: mol_type = other DNA
cctttgcggacagttg

SEQ ID NO: 12 - DNA, 16 bp, synthetic construct
  misc_feature 1..16: note = nucleic acid agent
  source 1..16: mol_type = other DNA
ctgatgacaggagtta

SEQ ID NO: 13 - DNA, 20 bp, synthetic construct
  misc_feature 1..20: note = primer
  source 1..20: mol_type = other DNA
atcttgtggaaaggacgaaa

SEQ ID NO: 14 - DNA, 23 bp, synthetic construct
  misc_feature 1..23: note = primer
  source 1..23: mol_type = other DNA
gccttattttaacttgctatttc

SEQ ID NO: 15 - DNA, 16 bp, synthetic construct
  source 1..16: mol_type = other DNA
cataatctgggaacga

SEQ ID NO: 16 - DNA, 16 bp, synthetic construct
  source 1..16: mol_type = other DNA
gtgtggttggaagcta

(19) United States
(12) Patent Application Publication
(10) Pub. No.: US 2025/0002914 A1
(43) Pub. Date: Jan. 2, 2025

(54) NUCLEIC ACID AGENTS FOR TREATMENT OF NON-SMALL-CELL LUNG CANCER

(71) Applicant: UNIVERSITY COLLEGE DUBLIN, Dublin (IE)

(72) Inventors: Rory JOHNSON, Bern (CH); Roberta ESPOSITO, Bern (CH); Taisia POLIDORI, Bern (CH)

(73) Assignee: UNIVERSITY COLLEGE DUBLIN, Dublin (IE)

(21) Appl. No.: 18/701,230

(22) PCT Filed: Oct. 7, 2022
(86) PCT No.: PCT/EP2022/077995

§ 371 Date: Apr. 14, 2024

(30) Foreign Application Priority Data
Oct. 13, 2021 (EP) 21202363.4

(51) Int. Cl. C12N 15/113

(52) U.S. Cl. CPC C12N 15/113; C12N 2310/113; C12N 2310/315; C12N 2310/3231; C12N 2320/31

(57) ABSTRACT

One aspect of the invention relates to a nucleic acid agent targeting a long non-coding RNA target selected from ENSG00000253616 (CHiLL1; SEQ ID NO 001) and ENSG00000272808 (CHiLL2; SEQ ID NO 002) for use in treatment or prevention of recurrence of non-small-cell lung cancer.

In another aspect, the invention relates to a pharmaceutical composition comprising a first nucleic acid agent targeting CHiLL1 and a second nucleic acid agent targeting CHiLL2.

Particular embodiments provide for the agents and compositions provided in treatment of cancers characterized by KRAS activating mutations, and/or drug resistance. Other particular embodiments provide their combination with platinum anticancer drugs.

The present invention relates to nucleic acid agents for treatment of carcinoma, particularly Non-small-cell lung cancer (NSCLC), to pharmaceutical compositions comprising such nucleic acid agents, and to methods for treating carcinoma, particularly NSCLC.

This application claims the benefit of priority of EP application 21202363.4, submitted 13 Oct. 2021, which is incorporated by reference herein in its entirety.

The experimental work underlying the present invention was published in Esposito et al., Cell Genomics Volume 2, Issue 9, 14 Sep. 2022, 100171, which is incorporated herein by reference.

### BACKGROUND OF THE INVENTION

Amongst the greatest unmet needs in oncology is lung cancer, the leading cause of cancer mortality worldwide (˜25%), and its most common subtype, Non-Small Cell Lung Cancer (NSCLC). Although the therapeutic landscape has changed substantially in recent years, metastatic NSCLC continues to have poor prognosis (5-year survival ˜20%). Targeted small-molecule therapies are only available for the few patients with EGFR (˜15% patients) and ALK mutations (˜3%). A minority of tumours are highly immunogenic and can be treated with immunotherapy alone. The remaining majority, including the 30% with activating KRAS mutations (KRAS-mut), receive cytotoxic platinum-based chemotherapeutics in combination with immunotherapy. Both targeted and non-targeted therapies are rapidly inactivated by resistance and few patients survive long-term.

A leading source of gene targets for nucleic acid-based therapeutics are the recently-discovered long non-coding RNAs (lncRNAs). LncRNAs' tumour-specific activity and patient-specific expression create potential for low side-effect, personalised therapies. With their total population likely to exceed 100,000, of which >98% remain uncharacterised, lncRNAs present a fertile untapped source of novel therapeutic targets.

Despite this promise, lncRNAs present significant hurdles to therapeutic screening, including poor annotation (Uszczynska-Ratajczak et al., 2018) and insensitivity to RNAi perturbation. A breakthrough arrived with CRISPR-Cas technology, which enables effective and scalable loss of function perturbations. Using a pooled library of single guide RNA (sgRNA) vectors, the entire expressed lncRNA population can be perturbed in an in vitro cell model. Screens identify hits based on cell-level phenotypes, making them compatible with established models of cancer hallmarks such as proliferation/fitness, chemosensitivity and migration.

Based on the above-mentioned state of the art, the objective of the present invention is to provide means and methods to better treat carcinoma, particularly NSCLC. This objective is attained by the subject-matter of the independent claims of the present specification, with further advantageous embodiments described in the dependent claims, examples, figures and general description of this specification.

Ginn et al. (2020), NON-CODIN RNA 6(3), 25:1-24, Jiang et al. (2021) Frontiers in Oncology 11, Article 761582; Sun et al. (2019) Frontiers in Pharmacology 10, Article 1457; and Tian (2021) Cancer Biology and Medicine 18(3) 675-692 all discuss the role of long non-coding RNA in lung cancer.

### SUMMARY OF THE INVENTION

One aspect of the invention relates to a nucleic acid agent targeting a long non-coding RNA target selected from ENSG00000253616 (CHiLL1; SEQ ID NO 001) and ENSG00000272808 (CHiLL2; SEQ ID NO 002, also referred to as GCAWKR in recent publications) for use in treatment or prevention of recurrence of non-small-cell lung cancer.

In another aspect, the invention relates to a pharmaceutical composition comprising a first nucleic acid agent targeting and capable of downregulating or inhibiting a long non-coding RNA target ENSG00000253616 (CHiLL1; SEQ ID NO 001) and a second nucleic acid agent targeting and capable of downregulating or inhibiting a long non-coding RNA target ENSG00000272808 (CHiLL2; SEQ ID NO 002).

Particular embodiments provide for the agents and compositions provided in treatment of cancers characterized by KRAS activating mutations, and/or drug resistance. Other particular embodiments provide their combination with platinum anticancer drugs.

### Terms and Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

The terms “comprising,” “having,” “containing,” and “including,” and other similar forms, and grammatical equivalents thereof, as used herein, are intended to be equivalent in meaning and to be open-ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictates otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic, and biochemical methods (see generally, Sambrook et al., Molecular Cloning: A Laboratory Manual, 4th ed. (2012) Cold Spring Harbor Laboratory Press, Cold Spring Harbor, N.Y. and Ausubel et al., Short Protocols in Molecular Biology (2002) 5th Ed, John Wiley & Sons, Inc.) and chemical methods.

The term “targets (a long non coding RNA)” in the context of the present specification relates to suppression or inhibition of the biological effect of the targeted lncRNA, by antisense oligonucleotides, including gapmers, RNAi or related mechanisms that lead to the physical degradation or otherwise mediated lack of biological function of the targeted RNA.

### General Molecular Biology: Nucleic Acid Sequences, Expression

The term gene refers to a polynucleotide that encodes a particular long non coding RNA after being transcribed. A polynucleotide sequence can be used to identify larger fragments or full-length coding sequences of the gene with which they are associated. Methods of isolating larger fragment sequences are known to those of skill in the art.

The term Nucleotides in the context of the present specification relates to nucleic acid or nucleic acid analogue building blocks, oligomers of which are capable of forming selective hybrids with RNA or DNA oligomers on the basis of base pairing. The term nucleotides in this context includes the classic ribonucleotide building blocks adenosine, guanosine, uridine (and ribosylthymine), cytidine, the classic deoxyribonucleotides deoxyadenosine, deoxyguanosine, thymidine, deoxyuridine and deoxycytidine. It further includes analogues of nucleic acids such as phosphotioates, 2′O-methylphosphothioates, peptide nucleic acids (PNA; N-(2-aminoethyl)-glycine units linked by peptide linkage, with the nucleobase attached to the alpha-carbon of the glycine) or locked nucleic acids (LNA; 2′0, 4′C methylene bridged RNA building blocks). Wherever reference is made herein to a hybridizing sequence, such hybridizing sequence may be composed of any of the above nucleotides, or mixtures thereof.

The terms capable of forming a hybrid or hybridizing sequence in the context of the present specification relate to sequences that under the conditions existing within the cytosol of a mammalian cell, are able to bind selectively to their target sequence. Such hybridizing sequences may be contiguously reverse-complimentary to the target sequence, or may comprise gaps, mismatches or additional non-matching nucleotides. The minimal length for a sequence to be capable of forming a hybrid depends on its composition, with C or G nucleotides contributing more to the energy of binding than A or T/U nucleotides, and on the backbone chemistry.

In the context of the present specification, the term hybridizing sequence encompasses a polynucleotide sequence comprising or essentially consisting of RNA (ribonucleotides), DNA (deoxyribonucleotides), phosphothioate deoxyribonucleotides, 2′-O-methyl-modified phosphothioate ribonucleotides, LNA and/or PNA nucleotide analogues. In certain embodiments, a hybridizing sequence according to the invention comprises 8, 9, 10, 11, 12, 13, 14, 15, 16, 17, 18, 19, 20, 21, 22, 23, 24, 25, 26, 27, 28, 29 or 30 nucleotides. In certain embodiments, the hybridizing sequence is at least 80% identical, more preferred 85%, 90%, 92%, 94%, 95%, 96%, 97%, 98% or 99% identical to the reverse complimentary sequence of SEQ ID 1 or SEQ ID 2. In certain embodiments, the hybridizing sequence comprises deoxynucleotides, phosphothioate deoxynucleotides, LNA and/or PNA nucleotides or mixtures thereof.

The term antisense oligonucleotide in the context of the present specification relates to an oligonucleotide having a sequence substantially complimentary to, and capable of hybridizing to, an RNA. Antisense action on such RNA will lead to modulation, particular inhibition or suppression of the RNA's biological effect. If the RNA is an mRNA, expression of the resulting gene product is inhibited or suppressed. Antisense oligonucleotides can consist of DNA, RNA, nucleotide analogues and/or mixtures thereof. The skilled person is aware of a variety of commercial and non-commercial sources for computation of a theoretically optimal antisense sequence to a given target. Optimization can be performed both in terms of nucleobase sequence and in terms of backbone (ribo, deoxyribo, analogue) composition. Many sources exist for delivery of the actual physical oligonucleotide, which generally is synthesized by solid state synthesis.

The term gapmer refers to a short DNA antisense oligonucleotide structure with RNA-like segments on both sides of the sequence, which are typically composed of locked nucleic acids (LNA), 2′-OMe, or 2′-F modified bases. Gapmers often comprise nucleotides modified with phosphorothioate (PS) groups, particularly in their 5′ and 3′ terminal regions. Gapmers are designed to hybridize to a target piece of RNA and silence the gene through the induction of RNase H cleavage. Binding of the gapmer to the target has a higher affinity due to the modified RNA flanking regions, as well as resistance to degradation by certain nucleases. Gapmers are being developed as therapeutics for a variety of cancers, viruses, and other chronic genetic disorders.

The term siRNA (small/short interfering RNA) in the context of the present specification relates to an RNA molecule capable of interfering with the expression (in other words: inhibiting or preventing the expression) of a gene comprising a nucleic acid sequence complementary or hybridizing to the sequence of the siRNA in a process termed RNA interference. The term siRNA is meant to encompass both single stranded siRNA and double stranded siRNA. siRNA is usually characterized by a length of 17-24 nucleotides. Double stranded siRNA can be derived from longer double stranded RNA molecules (dsRNA). According to prevailing theory, the longer dsRNA is cleaved by an endo-ribonuclease (called Dicer) to form double stranded siRNA. In a nucleoprotein complex (called RISC), the double stranded siRNA is unwound to form single stranded siRNA. RNA interference often works via binding of an siRNA molecule to the mRNA molecule having a complementary sequence, resulting in degradation of the mRNA. RNA interference is also possible by binding of an siRNA molecule to an intronic sequence of a pre-mRNA (an immature, non-spliced mRNA) within the nucleus of a cell, resulting in degradation of the pre-mRNA.

The term shRNA (small hairpin RNA) in the context of the present specification relates to an artificial RNA molecule with a tight hairpin turn that can be used to silence target gene expression via RNA interference (RNAi).

The term miRNA (microRNA) in the context of the present specification relates to a small non-coding RNA molecule (containing about 22 nucleotides) that functions in RNA silencing and post-transcriptional regulation of gene expression.

The term nucleic acid expression vector in the context of the present specification relates to a plasmid, a viral genome or an RNA, which is used to transfect (in case of a plasmid or an RNA) or transduce (in case of a viral genome) a target cell with a certain gene of interest, or -in the case of an RNA construct being transfected- to translate the corresponding protein of interest from a transfected mRNA. For vectors operating on the level of transcription and subsequent translation, the gene of interest is under control of a promoter sequence and the promoter sequence is operational inside the target cell, thus, the gene of interest is transcribed either constitutively or in response to a stimulus or dependent on the cell's status. In certain embodiments, the viral genome is packaged into a capsid to become a viral vector, which is able to transduce the target cell.

As used herein, the term pharmaceutical composition refers to a nucleic acid agent or pharmaceutical combination of the invention, or a pharmaceutically acceptable salt thereof, together with at least one pharmaceutically acceptable carrier. In certain embodiments, the pharmaceutical composition according to the invention is provided in a form suitable for topical, parenteral or injectable administration.

As used herein, the term pharmaceutically acceptable carrier includes any solvents, dispersion media, coatings, surfactants, antioxidants, preservatives (for example, antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington: the Science and Practice of Pharmacy, ISBN 0857110624). As used herein, the term treating or treatment of any disease or disorder (e.g. cancer) refers in one embodiment, to ameliorating the disease or disorder (e.g. slowing or arresting or reducing the development of the disease or at least one of the clinical symptoms thereof). In another embodiment “treating” or “treatment” refers to alleviating or ameliorating at least one physical parameter including those which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. Methods for assessing treatment and/or prevention of disease are generally known in the art, unless specifically described hereinbelow.

### DETAILED DESCRIPTION OF THE INVENTION

A first aspect of the invention relates to a nucleic acid agent targeting and capable of downregulating or inhibiting a long non-coding RNA target selected from
a. ENSG00000253616 (CHiLL1; SEQ ID NO 001)
b. ENSG00000272808 (CHiLL2; SEQ ID NO 002)
for use in treatment or prevention of recurrence of non-small-cell lung cancer.

In particular embodiments, the nucleic acid agents according to the invention are provided for use in treatment or prevention of recurrence of lung adenocarcinoma.

The evidence provided in the examples of the present specification supports the efficacy of the nucleic acid agents according to the invention as specified herein both in lung adenocarcinoma (LUAD) and large cell carcinoma, thus providing support for NSCLC as the umbrella indication.

One alternative of this first aspect of the invention relates to a nucleic acid agent that targets ENSG00000253616 (SEQ ID NO 001).

CHiLL1 is characterized as follows:
Gene ID: ENSG00000253616; Gene location: chr8:23,071,377-23,074,488 (-strand) (GRCh38 assembly coordinates)

In particular embodiments, the agent targeting CHiLL1 is provided for use in any type of carcinoma showing a higher than normal expression of CHiLL1.

Another alternative of this first aspect of the invention relates to a nucleic acid agent that targets ENSG00000272808 (SEQ ID NO 002).

CHiLL2 is characterized as follows
Gene ID: ENSG00000272808; Gene location: chr15:100,849,831-100,876,836 (+strand) (GRCh38 assembly coordinates)

Yet another particular alternative of this first aspect of the invention relates to a combination of two nucleic acid agents, one of which targets CHiLL1 and the other one of which targets CHiLL2.

### Antisense Oligonucleotides

In certain embodiments, the nucleic acid agent for use according to any one of the preceding aspects of the invention in any of their alternatives is an antisense oligonucleotide that is capable of hybridizing to its target CHiLL 1 or CHiLL 2. Antisense technology, in other words the administration of a short (10 to 35) oligomer of nucleotides, nucleotide analogues or a mix thereof, wherein the oligomer is capable of specifically hybridizing to its target sequence, will forcefully abrogate the biological function of the target RNA, as illustrated in the examples herein.

In certain embodiments, the nucleic acid agent according to any of the above aspects and embodiments comprises or consists of deoxyribonucleotides. Being a natural component of DNA, deoxyribonucleotides pose no issues of toxicity.

In certain embodiments, the nucleic acid agent according to any of the above aspects and embodiments comprises or consists of ribonucleotides. Being a natural component of RNA, deoxyribonucleotides pose no issues of toxicity, however the ubiquity of RNA degrading enzymes in the body require special technology for their delivery in most applications, such as, for example, liposomal delivery or their production in situ from a viral or plasmid vector.

The invention thus also encompasses a nucleic acid expression vector from which the nucleic acid agent according to the invention can be expressed under control of a promoter operable in a human cell. In particular embodiments, this can be a DNA vector, an RNA vector, a plasmid, a lentivirus vector, an adenoviral vector, and a retrovirus vector. In certain embodiments, the promoter is an EF-1 promoter, a CMV IE gene promoter, an EF-la promoter, a ubiquitin C promoter, or a phosphoglycerate kinase (PGK) promoter.

The invention further encompasses a retron construct that generates the DNA via reverse transcription of an RNA template encoding the antisense oligonucleotide sequence.

In certain embodiments, the nucleic acid agent according to any of the above aspects and embodiments comprises phosphorothioate bonds connecting ribonucleoside units, dexoxyribonucleoside units and/or nucleoside analogue units. Thiophophate (phosphorothioate) bonds offer higher resistance to degradation of oligonucleotides by phosphatases; toxicity issues, however, can be associated to the administration of high doses of thioate oligonucleotides.

In particular embodiments, the nucleic acid agent consists of dexoxyribonucleoside units and nucleoside analogue units connected by phosphorothioate bonds.

In certain embodiments, the nucleic acid agent for use according to any one of the preceding aspects of the invention in any of their alternatives comprises LNA (2′O, 4′C methylene bridged RNA building blocks). LNA hybrids with their complementary RNA or DNA are particularly stable and resistant to degradation.

In certain embodiments, the nucleic acid agent for use according to any one of the preceding aspects of the invention in any of their alternatives is an antisense gapmer.

RNA Agents and Nucleic Acid Agents from which RNA Agents can be Transcribed

In certain embodiments, the nucleic acid agent for use according to any one of the aspects of the nucleic acid agent is an RNA agent (oligoribonucleotide) capable of inhibiting or degrading the target, particularly an siRNA, a miRNA or an shRNA. Such RNA agents are well known in the art and their structural requirements have been explored.

In certain embodiments, the nucleic acid agent for use according to any one of the aspects of the nucleic acid agent encodes an oligoribonucleotide capable of inhibiting or degrading the target, particularly an siRNA, a miRNA or an shRNA.

In particularly embodiments, this siRNA, a miRNA or an shRNA will target an exon (post-processing) region of CHiLL1 or CHiLL2. The skilled person is aware that if the agent is designed to work by RNA interference or a related mechanism, the agent will favourable target an exon region of the gene, as—in contrast to antisense oligonucleotide agents—RNAi tends to work on the level of processed RNA.

### Exemplary Specific Sequences

In particular embodiments, the target is ENSG00000253616 (SEQ ID NO 001) and the nucleic acid agent comprises or consists of the sequence CAGGAGAAAAGCACAC (SEQ ID NO 003).

In particular embodiments, the target is ENSG00000253616 (SEQ ID NO 001) and the nucleic acid agent comprises or consists of the sequence ATTCTGGGTCACTGCT (SEQ ID NO 004).

In particular embodiments, the target is ENSG00000272808 (SEQ ID NO 002) and the nucleic acid agent comprises or consists of the sequence CATAATCTGGGAACGA (SEQ ID NO 005).

In particular embodiments, the target is ENSG00000272808 (SEQ ID NO 002) and the nucleic acid agent comprises or consists of the sequence GTGTGGTTGGAAGCTA (SEQ ID NO 006).

The cited oligonucleotide sequences are exemplary only and shall not limit the invention. SEQ ID NO 003 and 005 target an exon region of their respective target lncRNA. A particular set of embodiments of the invention provides nucleic acid agents targeting exons of SEQ ID 001 or 002, including regions covering exon-exon junctions (where the intron has been excised).

A particular set of embodiments of the invention provides antisense oligomer nucleic acid agents targeting introns of SEQ ID 001 or 002, including regions covering intron-exon junctions.

### Pharmaceutical Compositions

Another aspect of the invention relates to a pharmaceutical composition comprising a nucleic acid agent targeting and capable of downregulating or inhibiting a long non-coding RNA target CHiLL1 (ENSG00000253616; SEQ ID NO 001).

Another alternative of this aspect of the invention relates to a pharmaceutical composition comprising a nucleic acid agent targeting and capable of downregulating or inhibiting a long non-coding RNA target CHiLL2 (ENSG00000272808; SEQ ID NO 002).

A particular alternative aspect of the invention relates to a pharmaceutical composition comprising a first nucleic acid agent targeting and capable of downregulating or inhibiting CHiLL1, and a second nucleic acid agent targeting and capable of downregulating or inhibiting CHiLL2.

As shown in the examples, the combination of CHiLL1 and CHiLL2 targeting is synergetic beyond the mere addition of the effects of inhibiting each target individually.

Furthermore, additional inhibition of other tier 2 targets will confer further improvement of the effect. Thus, each of CHiLL1 and/or CHiLL2 targeting agents, or their combination, might further be favourable combined with agents targeting a member selected from the group comprising ENSG00000225880 (candidate 205), ENSG00000287114 (candidate 215) and ENSG00000188825 (candidate 507).

In certain embodiments, the nucleic acid agents targeting CHiLL1 and/or CHiLL2 comprised in the pharmaceutical compositions disclosed here are antisense oligomers.

In certain embodiments, such antisense oligomers targeting CHiLL1 and/or CHiLL2 comprised in the pharmaceutical compositions disclosed here comprise phosphorothioate bonds, and/or nucleoside analogues for increased stability or other pharmacologically advantageous modifications.

In particular embodiments thereof, the first nucleic acid agent and the second nucleic acid agent consist of dexoxyribonucleoside units and nucleoside analogue units connected by phosphorothioate bonds.

In particular embodiments thereof, the first nucleic acid agent and the second nucleic acid agent comprise LNA (2′0, 4′C methylene bridged RNA building blocks).

In particular embodiments thereof, that may be combined with any of the preceding embodiments, the first nucleic acid agent comprises or consists of a sequence selected from CAGGAGAAAAGCACAC (SEQ ID NO 003) and ATTCTGGGTCACTGCT (SEQ ID NO 004); and the second nucleic acid agent comprises or consists of a sequence selected from CATAATCTGGGAACGA (SEQ ID NO 005) and GTGTGGTTGGAAGCTA (SEQ ID NO 006).

If one or more of candidates 205, 215 or 507 are to be targeted, exemplary sequences for doing so include
for candidate 205, a sequence selected from CAGAAGCACGAGGGTT (SEQ ID NO 007) and AAGCTGAACCTGACAC (SEQ ID NO 008);
for candidate 215, a sequence selected from TCGTCCAGCTAATAAT (SEQ ID NO 009) and TTGGACAGAGTAAGCA (SEQ ID NO 010);
for candidate 507, a sequence selected from CCTTTGCGGACAGTTG (SEQ ID NO 011) and CTGATGACAGGAGTTA (SEQ ID NO 012).

In certain embodiments, the pharmaceutical composition according any of the preceding aspects or embodiments, is provided for use in treatment or prevention of recurrence of carcinoma.

In certain embodiments, the pharmaceutical composition according any of the preceding aspects or embodiments, is provided for use in treatment or prevention of recurrence of adenocarcinoma.

In certain particular embodiments, the pharmaceutical composition according any of the preceding aspects or embodiments, is provided for use in treatment or prevention of recurrence of non-small-cell lung cancer.

In certain more particular embodiments, the pharmaceutical composition according any of the preceding aspects or embodiments, is provided for use in treatment or prevention of recurrence of lung adenocarcinoma.

Administration to Patients Diagnosed with a Tumour Characterized by an Activating KRAS Mutation

Another aspect of the invention relates to the use of the nucleic acid agent, or the pharmaceutical composition, according to any of the above aspects and embodiments, by administration to a patient diagnosed with a tumour characterized by an activating KRAS mutation.

KRAS mutations are an important class of tumours, representing about ⅓ of NSCLC. Kempf et al. (European Respiratory Review 2016 25: 71-76; DOI: 10.1183/16000617.0071-2015) give a good review of the matter.

EGFR-positive lung cancer represents about 10-15% of lung cancer in the United States and generally appears in adenocarcinoma subtype of non-small cell lung cancer. Patients with lung cancers with EGFR mutations tend to have minimal to no smoking history. The evidence provided in the examples of the present specification supports the efficacy of the nucleic acid agents according to the invention as specified herein in EGFR mutated lung cancer. Ellison et al. (J Clin Pathol 2013; 66:79-89. doi:10.1136/jclinpath-2012-201194) review methods for testing for EGFR mutations in lung cancer.

### Administration to Patients Whose Tumour is Characterized by CHiLL1 or CHiLL2 Overexpression

Another aspect of the invention relates to the use of the nucleic acid agent, or the pharmaceutical composition, according to any of the above aspects and embodiments, by administration to a patient diagnosed with a tumour characterized by overexpression of CHiLL1 and/or overexpression of CHiLL2.

Overexpression can be determined by quantitative RT PCR relative to a panel of tumour control samples or to tissue of the same origin as the tumour (e.g. lung tissue) of the same patient or a panel of healthy control samples, and can be normalized relative to a household gene for the individual sample.

Administration to patients whose tumour is characterized by drug resistance Another aspect of the invention relates to the use of the nucleic acid agent, or the pharmaceutical composition, according to any of the above aspects and embodiments, by administration to a patient diagnosed with a tumour characterized by resistance. i.e. lack of adequate or expected response to chemotherapy. Chemotherapy in this context relates to treatment with a cytotoxic or cytostatic drug commonly used in the treatment of the cancer.

In certain embodiments, the agent or pharmaceutical composition is administered to a patient whose tumour is characterized by resistance to a drug comprising a platinum-containing complex.

In certain particular embodiments, the agent or pharmaceutical composition is administered to a patient whose tumour is characterized by resistance to a platinum-containing drug selected from carboplatin, satraplatin, cisplatin, dicycloplatin, nedaplatin, oxaliplatin, picoplatin, and/or triplatin tetranitrate.

Combination Medication with Platinum Anticancer Drugs

Another aspect of the invention relates to the use of the nucleic acid agent, or the pharmaceutical composition, according to any of the above aspects and embodiments, in combination with a drug comprising a platinum-containing complex.

Particular embodiments of this aspect include the combination with a platinum-containing drug selected from carboplatin, satraplatin, cisplatin, dicycloplatin, nedaplatin, oxaliplatin, picoplatin, and/or triplatin tetranitrate.

More particular embodiments of this aspect include the combination with a platinum-containing drug selected from carboplatin and cisplatin.

### Medical Treatment

Similarly, within the scope of the present invention is a method or treating cancer, particularly NSCLC, in a patient in need thereof, comprising administering to the patient a nucleic acid agent or pharmaceutical combination according to the above description.

Pharmaceutical Compositions. Administration/Dosage Forms and Salts

According to one aspect of the compound according to the invention, the nucleic acid agent or pharmaceutical combination according to the invention is provided as a pharmaceutical composition, pharmaceutical administration form, or pharmaceutical dosage form.

In certain embodiments of the invention, the nucleic acid agent or pharmaceutical combination of the present invention is typically formulated into pharmaceutical dosage forms to provide an easily controllable dosage of the drug and to give the patient an easily handleable product.

Similarly, a dosage form for the prevention or treatment of cancer, particularly NSCLC, is provided, comprising a nucleic acid agent or pharmaceutical combination according to any of the above aspects or embodiments of the invention.

Certain embodiments of the invention relate to a dosage form for parenteral administration, such as subcutaneous, intravenous, intrahepatic or intramuscular injection forms. Optionally, a pharmaceutically acceptable carrier and/or excipient may be present.

The dosage regimen for the compounds of the present invention will vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. In certain embodiments, the compounds of the invention may be administered in a single daily dose, or the total daily dosage may be administered in divided doses of two, three, or four times daily.

The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The pharmaceutical compositions of the present invention can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc. They may be produced by standard processes, for instance by conventional mixing, granulating, dissolving or lyophilizing processes. Many such procedures and methods for preparing pharmaceutical compositions are known in the art, see for example L. Lachman et al. The Theory and Practice of Industrial Pharmacy, 4th Ed, 2013 (ISBN 8123922892).

### Method of Manufacture and Method of Treatment According to the Invention

The invention further encompasses, as an additional aspect, the use of a nucleic acid agent or pharmaceutical combination as identified herein, for use in a method of manufacture of a medicament for the treatment or prevention of carcinoma, particularly adenocarcinoma, more particularly NSCLC, as further specified herein.

Similarly, the invention encompasses methods of treatment of a patient having been diagnosed with cancer, particularly NSCLC, as further specified herein. This method entails administering to the patient an effective amount of a nucleic acid agent or pharmaceutical combination as identified herein.

Wherever alternatives for single separable features are laid out herein as “embodiments”, it is to be understood that such alternatives may be combined freely to form discrete embodiments of the invention disclosed herein.

The invention further encompasses the following items:

Item 1. A nucleic acid agent targeting and capable of downregulating or inhibiting a long non-coding RNA target selected from
a. ENSG00000253616 (CHiLL1; SEQ ID NO 001)
b. ENSG00000272808 (CHiLL2; SEQ ID NO 002)
for use in treatment or prevention of recurrence of non-small-cell lung cancer, particularly for use in treatment or prevention of recurrence of lung adenocarcinoma.

Item 2. The nucleic acid agent for use according to item 1, wherein the target is ENSG00000253616 (SEQ ID NO 001).

Item 3. The nucleic acid agent for use according to item 1, wherein the target is ENSG00000272808 (SEQ ID NO 002).

Item 4. The nucleic acid agent for use according to any one of the preceding items, wherein the agent is an antisense oligonucleotide.

Item 5. The nucleic acid agent for use according to any one of the preceding items, wherein the nucleic acid agent comprises deoxyribonucleotides.

Item 6. The nucleic acid agent for use according to any one of the preceding items, wherein the nucleic acid agent comprises phosphorothioate bonds connecting ribonucleoside units, dexoxyribonucleoside units and/or nucleoside analogue units, particularly wherein the nucleic acid agent consists of dexoxyribonucleoside units and nucleoside analogue units connected by phosphorothioate bonds.

Item 7. The nucleic acid agent for use according to any one of the preceding items, wherein the nucleic acid agent comprises LNA (2′O, 4′C methylene bridged RNA building blocks).

Item 8. The nucleic acid agent for use according to any one of the preceding items, wherein the target is ENSG00000253616 (SEQ ID NO 001) and wherein
a. the nucleic acid agent comprises or consists of the sequence

(SEQ ID NO 003)
CAGGAGAAAAGCACAC
or
b.
(SEQ ID NO 004)
ATTCTGGGTCACTGCT

Item 9. The nucleic acid agent for use according to any one of the preceding items 1 to 7, wherein the target is ENSG00000272808 (SEQ ID NO 002) and wherein
a. the nucleic acid agent comprises or consists of the sequence

(SEQ ID NO 005)
CATAATCTGGGAACGA
or
b.
(SEQ ID NO 006)
GTGTGGTTGGAAGCTA

Item 10. The nucleic acid agent for use according to any one of the preceding items 1 to 4, wherein the nucleic acid agent encodes an oligoribonucleotide capable of inhibiting or degrading the target, particularly wherein the agent encodes an siRNA, a miRNA or an shRNA.

Item 11. A pharmaceutical composition comprising
a. a first nucleic acid agent targeting and capable of downregulating or inhibiting a long non-coding RNA target ENSG00000253616 (CHiLL1; SEQ ID NO 001) and
b. a second nucleic acid agent targeting and capable of downregulating or inhibiting a long non-coding RNA target ENSG00000272808 (CHiLL2; SEQ ID NO 002).

Item 12. A pharmaceutical composition comprising at least one of, particularly both of,
a. a nucleic acid agent targeting and capable of downregulating or inhibiting a long non-coding RNA target ENSG00000253616 (CHiLL1; SEQ ID NO 001)
b. a nucleic acid agent targeting and capable of downregulating or inhibiting a long non-coding RNA target ENSG00000272808 (CHiLL2; SEQ ID NO 002).
   and said pharmaceutical composition further comprising at least one of
a nucleic acid agent targeting and capable of downregulating or inhibiting a long non-coding RNA target ENSG00000225880 (candidate 205);
a nucleic acid agent targeting and capable of downregulating or inhibiting a long non-coding RNA target ENSG00000287114 (candidate 215);
a nucleic acid agent targeting and capable of downregulating or inhibiting a long non-coding RNA target ENSG00000188825 (candidate 507).

Item 13. The pharmaceutical composition according to item 11 or 12, wherein the first nucleic acid agent and the second nucleic acid agent comprise phosphorothioate bonds connecting ribonucleoside units, dexoxyribonucleoside units and/or nucleoside analogue units,
particularly wherein the first nucleic acid agent and the second nucleic acid agent consist of dexoxyribonucleoside units and nucleoside analogue units connected by phosphorothioate bonds.

Item 14. The pharmaceutical composition according to any one of the preceding items 11-13, wherein the first nucleic acid agent and the second nucleic acid agent comprise LNA (2′O, 4′C methylene bridged RNA building blocks).

Item 15. The pharmaceutical composition according to any one of the preceding items 11-14, wherein
a. the first nucleic acid agent comprises or consists of a sequence selected from CAGGAGAAAAGCACAC (SEQ ID NO 003) and ATTCTGGGTCACTGCT (SEQ ID NO 004); and
b. the second nucleic acid agent comprises or consists of a sequence selected from CATAATCTGGGAACGA (SEQ ID NO 005) and GTGTGGTTGGAAGCTA (SEQ ID NO 006).

Item 16. The pharmaceutical composition according to any one of the preceding items 12-15, wherein
the nucleic acid agent targeting ENSG00000225880 (candidate 205) comprises or consists of a sequence selected from CAGAAGCACGAGGGTT (SEQ ID NO 007) and AAGCTGAACCTGACAC (SEQ ID NO 008);
the nucleic acid agent targeting ENSG00000287114 (candidate 215) comprises or consists of a sequence selected from TCGTCCAGCTAATAAT (SEQ ID NO 009) and TTGGACAGAGTAAGCA (SEQ ID NO 010)
the nucleic acid agent targeting ENSG00000188825 (candidate 507) comprises or consists of a sequence selected from CCTTTGCGGACAGTTG (SEQ ID NO 011) and CTGATGACAGGAGTTA (SEQ ID NO 012).

Item 17. The pharmaceutical composition according to any one of the preceding items 11 to 16 for use for use in treatment or prevention of recurrence of non-small-cell lung cancer,
particularly for use in treatment or prevention of recurrence of lung adenocarcinoma.

Item 18. The nucleic acid agent or the pharmaceutical composition for use according to any one of the preceding items 1 to 15, wherein the nucleic acid agent or the pharmaceutical composition is administered to a patient diagnosed with a tumour characterized by an activating KRAS mutation.

Item 19. The nucleic acid agent or the pharmaceutical composition for use according to any one of the preceding items 1 to 16, wherein the nucleic acid agent or the pharmaceutical composition
a. targets CHiLL1 and the tumour is characterized by overexpression of CHiLL1 and/or
b. targets CHiLL2 and the tumour is characterized by overexpression of CHiLL2.

Item 20. The nucleic acid agent or the pharmaceutical composition for use according to any one of the preceding items 1 to 17, wherein the agent or pharmaceutical composition is administered to a patient diagnosed with a tumour characterized by resistance to chemotherapy,
particularly wherein the tumour is characterized by resistance to a drug comprising a platinum-containing complex,
more particularly wherein the tumour is characterized by resistance to a platinum-containing drug selected from carboplatin, satraplatin, cisplatin, dicycloplatin, nedaplatin, oxaliplatin, picoplatin, and/or triplatin tetranitrate.

Item 21. The nucleic acid agent or the pharmaceutical composition for use according to any one of the preceding items 1 to 19, wherein the nucleic acid agent or pharmaceutical composition is administered in combination with a drug comprising a platinum-containing complex,
particularly in combination with a platinum-containing drug selected from carboplatin, satraplatin, cisplatin, dicycloplatin, nedaplatin, oxaliplatin, picoplatin, and/or triplatin tetranitrate,
more particularly wherein the nucleic acid agent is administered in combination with a drug selected from carboplatin and cisplatin.

The invention is further illustrated by the following examples and figures, from which further embodiments and advantages can be drawn. These examples are meant to illustrate the invention but not to limit its scope.

### DESCRIPTION OF THE FIGURES

FIG. 1. Multi-hallmark CRISPR discovery of lncRNAs promoting non-small cell lung cancer. A) CRISPR-deletion pooled screening strategy for lncRNAs promoting NSCLC hallmarks. B) Gene annotations used for candidates' selection. Numbers indicate lncRNA gene loci. C) Expression of targeted lncRNAs in A549 cells. D) Library composition, in terms of targeted regions. Note that some lncRNA loci are represented by >1 targeted transcription start site (TSS).

FIG. 2. Adapting CRISPR screens to cancer hallmarks. A) Proliferation: The strategy employs complementary negative (drop-out) (growth-promoting lncRNAs' pgRNAs are depleted) and positive (CFSE dye) (growth-promoting lncRNAs' pgRNAs are enriched) formats. B) Cisplatin sensitivity: Another complementary strategy is employed. In the negative (drop-out) “survival” screen, cells are exposed to high cisplatin doses (IC30, IC80). Resistance-promoting lncRNAs' pgRNAs will be depleted in surviving cells. In the positive “death” screen, cells that die in response to low cisplatin concentration (IC20) are collected, and enriched pgRNAs identify resistance-promoting lncRNAs. C) Migration: Cells that are capable/incapable of migrating through a porous membrane over a given time period are separately collected. Migration-promoting lncRNAs are identified via their pgRNAs' enrichment in migration-impaired cells. D) LncRNA candidates, neutral and positive controls, ranked by P-value in A549 drop-out screen (statistical significance estimate using Wilcoxon test). E) A549 drop-out screen. Horizontal line indicates cutoff for hits at FDR<0.2. Previously published lncRNAs in NSCLC and other cancers are labelled in green and blue, respectively. F) Comparison of A549 and H460 drop-out screens (statistical significance estimated using Pearson correlation). G) Comparison of A549 and H460 cisplatin survival screen (Pearson correlation). H) Numbers of screen hits at FDR<0.2

FIG. 3. Validation of screen hits shows reproducible phenotypes. A) Candidate_205 (left panel) and Candidate_509 (right panel) loci. Primers and ASO sites are indicated above. The TSS target region and the 10 pgRNAs from the screening library are indicated below. B) Normalised pgRNA counts over the course of the drop-out screen in A549. Arrows indicate pgRNAs that were cloned here for validation. C) Competition assay. Fluorescently-labelled cells carrying pgRNAs for control AAVS1 locus (green, GFP) or indicated targets (red, mCherry) were mixed in equal proportions and their subsequent populations measured by flow cytometry. pgRNAs targeting the open reading frame of essential ribosomal protein RPS5 were used as positive control. Experiments were performed in biological triplicate. Error bars indicate standard deviation; statistical significance was estimated by Student's t test at the last timepoint. D) Antisense oligonucleotides were used to separately target the two lncRNAs sharing the TSS at Candidate_205. For each, two different ASOs were employed (1 and 2) in A549 cells. Upper panels: LINC00115; Lower panels: RP11-206L10.11. Left: cell population; right: RNA expression measured by RT-qPCR. Four biological replicates were performed. Error bars indicate standard deviation; significance was estimated by one-tailed Student's t test. E) Left: Normalised counts of pgRNAs in migrated and non-migrated cell populations from A549 migration screen. Right: Validation experiments with A549 cell migration across transwell supports over 24 h. Cells were treated with ASOs targeting indicated lncRNAs or a non-targeting control. F) Left: RT-qPCR in A549 cells treated with two distinct ASOs each for Candidate_215, 448 and 489. Expression was normalised to a non-targeting ASO. Right: Crystal violet quantification normalised to non-targeting ASO. Data are plotted as mean±SD from three independent biological replicates. Statistical significance was estimated by one-tailed Student's t test.

FIG. 4. Functional landscape of lncRNAs in NSCLC. A) Target prioritisation pipeline (TPP) integrates multiple screens to generate a unified hit ranking. TPP employs both effect size (Robust Rank Aggregation; RRA) and statistical significance (Brown's method). B) Comparing performance of screens and integration methods. Performance is measured as the area under the ROC curve (AUC) based on correctly recalling/rejecting library controls (positive controls and neutral controls, respectively). C) Ternary plot contains all significant hits (FDR<0.2) in pan-hallmark analysis (circles) or individual hallmarks (diamonds). The three corners each represent a hallmark, indicated by symbols. The proximity to each corner is driven by the TPP significance calculated for each individual hallmark. The candidates selected for further validations and selected protein-coding controls are indicated by library candidate number or gene name, respectively. Selected lncRNAs previously associated with LUAD are labelled with blue. Petal plots display the hallmark contributions of selected lncRNAs, and are explained in the panel to the left. D) The number of hits discovered in pan-hallmark and individual hallmark screens. Right: The target composition of the screening library for comparison. “Lung lncRNAs” indicate previously-published, functionally-validated lncRNAs in lung cancer (Vancura et al., 2021) E) Expression of pan-hallmark hits compared to all other screened lncRNAs (non-hits) in the KRAS-mutated samples from the TCGA LUAD cohort (n=101). Statistical significance was estimated by Welch's t-test, one-tailed. F) Expression of pan-hallmark hits compared to non-hits in an independent cohort of LUAD samples and healthy tissues (87 tumour; 77 normal) (Seo et al., 2012). Statistical significance: pairwise two-tailed Student's t-test. G) Pan-cancer recurrent amplifications and deletions, estimated in the Pan-Cancer Analysis of Whole Genomes Consortium (PCAWG) cohort. Statistical significance was estimated by Fisher's exact test.

FIG. 5. Therapeutic targeting of oncogenic lncRNAs CHiLL1 and CHiLL2. A) Left: Experimental workflow to test knockdown efficiency and phenotypic effect of ASO transfection. Right: Summary of all ASO/cell line results. Rows: Targeted lncRNAs; Columns: ASOs and cell lines. Values reflect the mean log 2 fold change in viability following ASO transfection, with respect to a control non-targeting ASO, from at least two independent biological replicates. Numbers to the left indicate library candidate identifiers, and are grouped into Tiers. Each lncRNA is targeted by two independent ASO sequences (1&2, below), which were previously validated for knockdown efficiency. MALAT1 lncRNA is used as positive control. “Normal” cells are non-transformed cells of lung origin. B) Expression of Tier 2 lncRNAs—CHiLL1, CHiLL2, Candidate_205 (LINC001150), Candidate_507 (LINC00910)—in TCGA RNA-seq. LUAD: 513 samples, Normal: 59 samples. Statistical significance estimated by t-test; **** P<0.0001. Data was not available for Candidate_215 in the TCGA dataset. C) Genomic loci encoding CHiLL1 (upper panel) and CHiLL2 (lower panel). D) A549 cell growth upon transfection with two independent ASOs. Results are normalised to non-targeting control ASO (n=4 biological replicates; error bars: standard deviation; statistical significance: two-tailed Student's t test). E) Left: Viability of H441 spheroid cultures seven days after transfection of 25 nM ASOs. mTOR ASO was used as positive control (n=4 biological replicates; error bars: standard deviation; one-tailed Student's t test). Right: Representative images (Leica DM IL LED Tissue Culture Microscope). F) Viability of BE874 organoids grown from a KRAS-mutant patient-derived xenograft after transfection of 25 nM CHiLL1 ASO (n=4 biological replicates; n>3 technical replicates; error bars: standard deviation; one-tailed Student's t test). G) ASO cocktails. Above: For all experiments, the total amount of ASO did not vary (25 nM). Cocktails were composed of equal proportions of indicated ASOs. For single ASO experiments, the best performing ASO for each target was used. Below: A549 cell populations, normalised to non-targeting ASO (n=4 biological replicates; error bars: standard deviation; statistical significance: one-tailed Mann Whitney test). H) Expression of Tier 2 lncRNAs in KRAS-mut LUAD tumours (TCGA). Each cell represents a patient, and is coloured to reflect expression as estimated by RNA-seq (with expression defined as >0.5 FPKM). Below, the percentage of patients with at least one lncRNA from the indicated cocktails.

FIG. 6. CHiLL1 and CHiLL2 drive distinct but overlapping oncogenic pathways. A) Genomic elements in CHiLL2 exons (grey rectangles) and introns. Strand is indicated by element direction, where appropriate. Plot was generated with ezTracks (Guillen-Ramirez and Johnson, 2021). B) Representative confocal microscopy images of RNA-FISH performed with CHiLL1 and CHiLL2 probe sets in A549 cells. Selected lncRNA foci are arrowed. C) Ratio of concentrations of indicated RNAs as measured by RT-qPCR in nuclear/cytoplasmic fractions of A549 cells. MALAT1 and GAPDH are used as nuclear and cytosolic controls, respectively. D) Expression of CHiLL1 as quantified in the three RNA-seq replicates from A549 RNA. The y-axis represents the normalized expression (counts) per nucleotide and the boxplots show the variance of inference using bootstraps generated by Kallisto. E) Fold change in gene expression in response to CHiLL1 knockdown by ASO1 and ASO2 in A549 cells. Statistical significance: Pearson correlation coefficient. Trendline depicts regression line. Numbers indicate genes in each quadrant. F) Statistical enrichment of Kyoto Encyclopedia of Genes and Genomes (KEGG) pathways amongst differentially expressed genes (DEGs) resulting from CHiLL1 knockdown. Only genes significantly affected in common by ASO1 and AS02 were included, for each cell line. Cancer-relevant pathways that are significant for both cell lines are highlighted in green. Statistical significance: Pearson correlation coefficient. G) Annexin-V apoptosis assay 24 h after transfection with ASOs targeting CHiLL1 (left) and CHiLL2 (right). H) As for C, for CHiLL2. I) As for D, but comparing knockdown of CHiLL2 with the same ASO in A549 and H460 cells (P=2.2e-16; R=0.8; statistical significance: Pearson correlation coefficient). J) Molecular Signatures Database (MSigDB) term enrichment significance for differentially-expressed genes in common between A549 and H460 cells. The most significant terms are shown. y-axis: Adjusted P-value (q-value). K) Cell cycle assay results after 24 h upon knock-down of CHiLL1 and CHiLL2 in A549. Cell cycle phases were determined according to the Dean-Jett Fox (DJF) model. (n=2 biological replicates, error bars: standard deviation; statistical significance: two-tailed Student's t test) L) Cell migration across transwell supports in A549 cells. Representative images of cells transfected with ASO control and CHiLL2 ASO and allowed to migrate across the membrane for 24 hours. The crystal violet quantification is normalised to non-targeting ASO (n=4 biological replicates, error bars: standard deviation; statistical significance: one-tailed Student's t test).

FIG. 7: A) Tumour volume of cohorts of NGS mice injected with A549 cells and treated (subcutaneous injections) with a scramble ASO (Control) or targeting ASO (CHiLL2-GCAWKR). Data are the means±s.d. of different technical replicates (N=3 in BR1 and N=4 in BR2). The lower line corresponds to the treatment, the upper to the control animals.
B) Weight variation of mice treated as described in A) and mice exposed daily to ASO control or ASO CHiLL2-GCAWKR in two independent biological replicates.

### EXAMPLES

The data provided herein relate to a study in which the inventors employed the inherent versatility of CRISPR screening to comprehensively map the functional landscape of lncRNAs in KRAS-mutant NSCLC across diverse cancer hallmarks and backgrounds. This resource identifies lncRNA vulnerabilities, which can be targeted by potent, on-target and low-toxicity ASO inhibitors to inhibit cell growth and sensitise them to existing chemotherapeutics. The inventors evaluate the potential for such ASOs in personalised therapy, and show how their modes of action can be evaluated by quantitative analysis of the cellular transcriptome.

### Example 1: A Versatile CRISPR Screening Pipeline for Long Non-Codinq RNAs in NSCLC

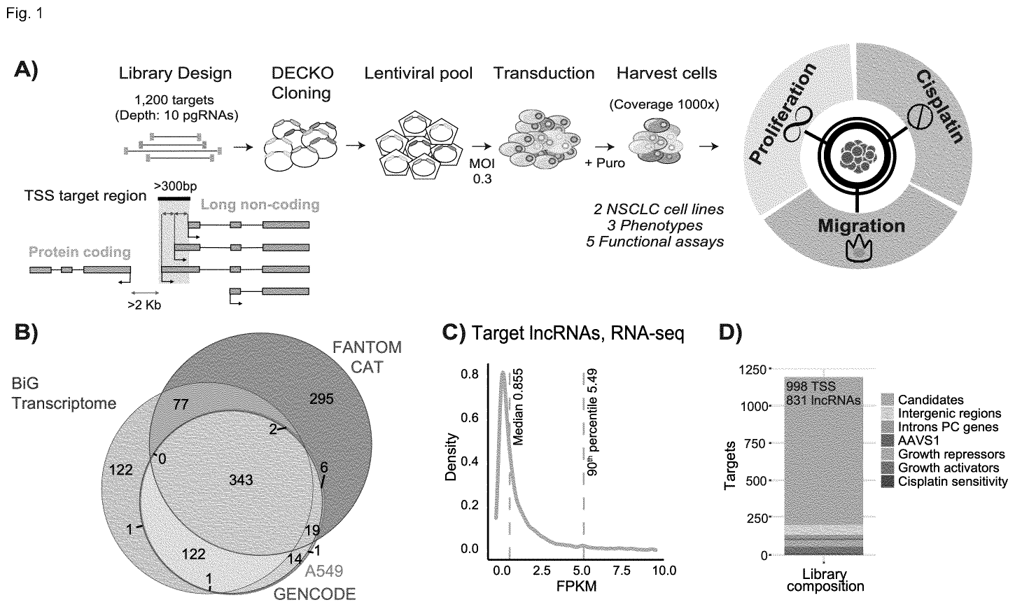

To identify lncRNAs promoting NSCLC, the inventors adapted the DECKO (dual excision CRISPR knock-out) CRISPR-deletion (CRISPR-del) system to high-throughput pooled format (Esposito et al., Cancer Cell 15, 545-557) (FIG. 1A). The inventors' approach achieves loss-of-function perturbations by deleting target genes' transcription start site (TSS) via paired guide RNAs (pgRNAs), demonstrated previously to effectively inhibit gene expression (Furlan et al., 2018; Mol. Cell 70, 462-472.e8).

The inventors created a screening library to comprehensively interrogate the NSCLC lncRNA transcriptome. The inventors integrated three published annotations (Frankish et al., 2019 Nucleic Acids Res. 47, D766-D773; Hon et al., 2017 Nature 543, 199-204; You et al., 2017 Genome Res. 27, 1050-1062) together with in-house transcriptome assembly from A549 NSCLC cells (FIG. 1B). From this merged annotation the inventors selected genes with detectable expression (>0.1 FPKM) that do not overlap protein-coding genes (PCGs), for a final target set of 831 lncRNAs (FIGS. 1C and 1D).

Effective gene perturbations depend on accurate identification of TSS. The inventors integrated evidence from CAGE (Hon et al., ibid.), DNasel hypersensitivity and promoter-like chromatin states from NSCLC cells (Davis et al., 2018 Nucleic Acids Res. 46, D794-D801; Dunham et al., 2012 Nature 489, 57-7) to identify 998 high-confidence TSS driving the above target genes, which are henceforth named “Candidate_1” and so on. To these the inventors added neutral control intergenic regions (not expected to influence cell phenotype) and positive control PCGs with known roles in cell proliferation and cisplatin resistance (FIG. 1D).

These targets form the basis for ‘libDECKO-NSCLC1’, a CRISPR-deletion library with a depth of 10 unique pgRNAs per target, comprising altogether 12,000 pgRNAs (FIG. 1D). The median deletion size is 1,512 bp. After cloning into the DECKO backbone, sequencing revealed high quality in terms of sequence identity (60.8% perfect match across both spacers) and coverage (90th to 10th percentile count ratios: 4.6-fold).

To identify hits of general relevance to NSCLC, the inventors performed parallel experiments in two widely-used NSCLC models, A549 and H460, both carrying activating KRAS mutations. Non-clonal cell lines were generated that stably express high-levels of Cas9 protein (Stojic et al., 2018 Nucleic Acids Res. 46, 5950-5966), as evidenced by blue fluorescent protein (BFP). Targeting known NSCLC-promoting lncRNA DNMBP-AS1 (Candidate_331), resulted in deletion of its promoter region and loss of expression (not shown), supporting the effectiveness of CRISPR-del for lncRNA loss of function.

Multi-Phenotype Mapping of NSCLC lncRNAs

Cancers thrive via a variety of phenotypic “hallmarks”. For practical reasons, previous CRISPR screens have been limited to a single hallmark, either proliferation or drug resistance, and focussed on a negative “drop-out” format, where pgRNAs for genes of interest are depleted.

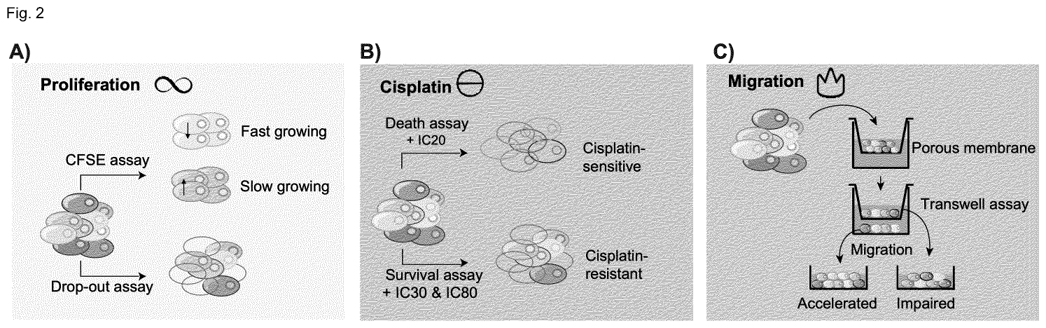

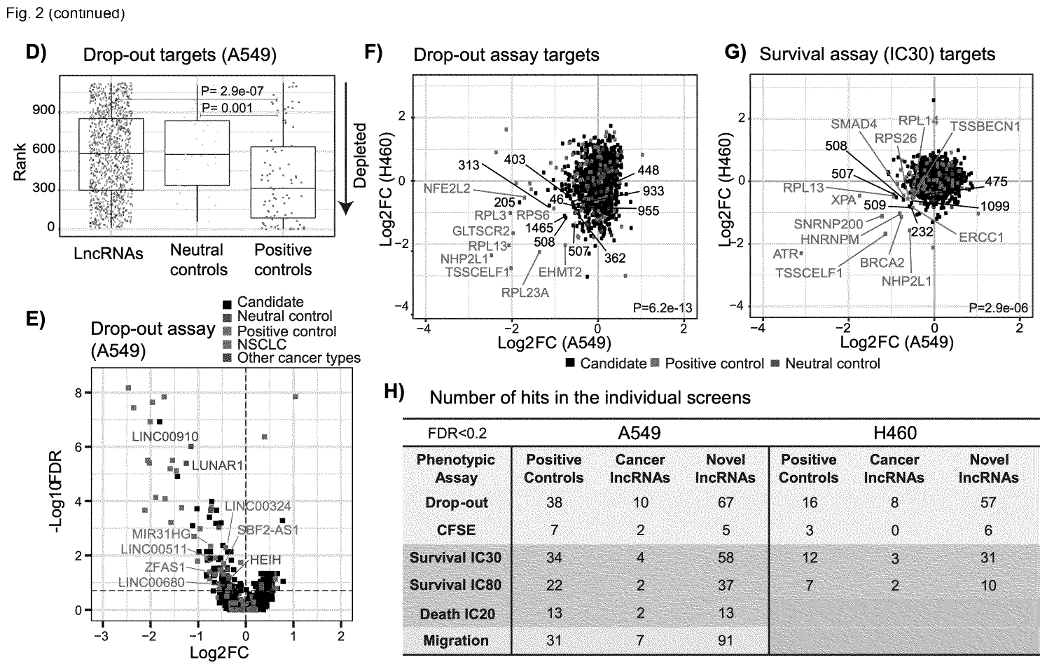

To generate a more comprehensive and biomedically-relevant resource of lncRNAs in Lung Adenocarcinoma (LUAD), the inventors leveraged the versatility of pooled screening to study three hallmarks: proliferation, chemo-resistance and invasion (FIGS. 2A, 2B and 2C). To boost sensitivity, the inventors implemented complementary “positive” screens, where pgRNAs of interest are enriched. To identify lncRNAs promoting cell fitness and proliferation, the inventors combined (i) a classical drop-out, where targets' pgRNAs become depleted, and (ii) a positive screen using CFSE (Carboxyfluorescein succinimidyl ester) dye to identify growth-promoting lncRNAs by enrichment of their pgRNAs in slow-growing cells (FIG. 2A).

As expected, pgRNAs targeting positive-control genes were significantly depleted in drop-out screens, while neutral controls were not (FIG. 2D). To enable to gauge the relative efficiency of the promoter deletion strategy, pgRNAs targeting positive-control protein-coding genes had been split between two distinct LOF modalities: (1) promoter-deletion, similar to lncRNAs; and (2) conventional open reading frame (ORF) mutation, expected to cause high-efficiency translational frameshift. Promoter-deletion pgRNAs displayed a detectable but lower phenotypic impact, reflecting the known lower efficiency of CRISPR-deletion, and indicating that a CRISPR-del screen for lncRNAs faces intrinsically lower sensitivity compared to ORF-targeting screens for PCGs.

Using biologically replicated drop-out screens, 77 lncRNAs were identified as necessary for proliferation of A549 cells. These include known NSCLC lncRNAs, such as LINC00324, ZFAS1, MIR31 HG, SBF2-AS1, LINC00680 and LINC00511, that was also found in H460. In addition, lncRNAs identified in other cancer types include LUNAR1, HEIH and LINC00910 (FIG. 2E).

The factors influencing pgRNA deletion efficiency are poorly understood. Using growth phenotype as a proxy for deletion efficiency, the inventors observed expected correlation between observed and bioinformatically-predicted sgRNA efficiency (RuleSet2 algorithm). On the other hand, the inventors found no relationship with pgRNA orientation, and a weak tendency for larger deletions to produce stronger phenotypes, possibly due to greater impact on lncRNA expression.

Next, the inventors compared equivalent drop-out screens in the two NSCLC backgrounds. There was a significant concordance amongst identified targets, supporting the broader disease relevance of most hits (FIG. 2F). To strengthen these data, the inventors performed complementary CFSE screens in the same cells. As expected, these positive screens displayed anti-correlation with drop-out results in H460 cells, although not in A549 cells, possibly for technical reasons.

Patients with KRAS-mutant tumours are usually treated with cytotoxic platinum-based chemotherapeutics, but tumours frequently evolve resistance. To identify lncRNAs promoting chemoresistance, the inventors employed complementary screens (FIG. 2B) at carefully chosen cisplatin concentrations. Again, correlated results were observed across cell backgrounds (FIG. 2G), and PCGs with known roles in cisplatin resistance were correctly identified (red points in FIG. 2G). Again, complementary survival (negative) and death (positive) screens were anti-correlated for high cisplatin dose (IC80), although not for low (IC30).

Migration is a key hallmark underlying invasion and metastasis of tumour cells. By isolating cells with rapid or slow migration through a porous membrane for 48 h (FIG. 2C), the inventors screened for migration-promoting lncRNAs (Seo et al., 2014). This yielded 98 lncRNAs, of which seven were already associated with migration and invasion in numerous cancer types, including NORAD, DANCR and SNHG29.

These data, summarized in FIG. 2H, represent a resource of functional lncRNAs in NSCLC hallmarks.

### Screen Hits can be Validated and Function Via RNA Products

To further ensure the reliability of these results, the inventors selected two lncRNA TSS for further validation, based on their top ranking and consistency between the two cell lines: Candidate_205, identified as top hit in drop-out screens (A549: Log2FC=−1.81, FDR=1.2e-07) and Candidate_509 in both proliferation and cisplatin (A549: Log2FC=−1.15, FDR=9.78e-07).

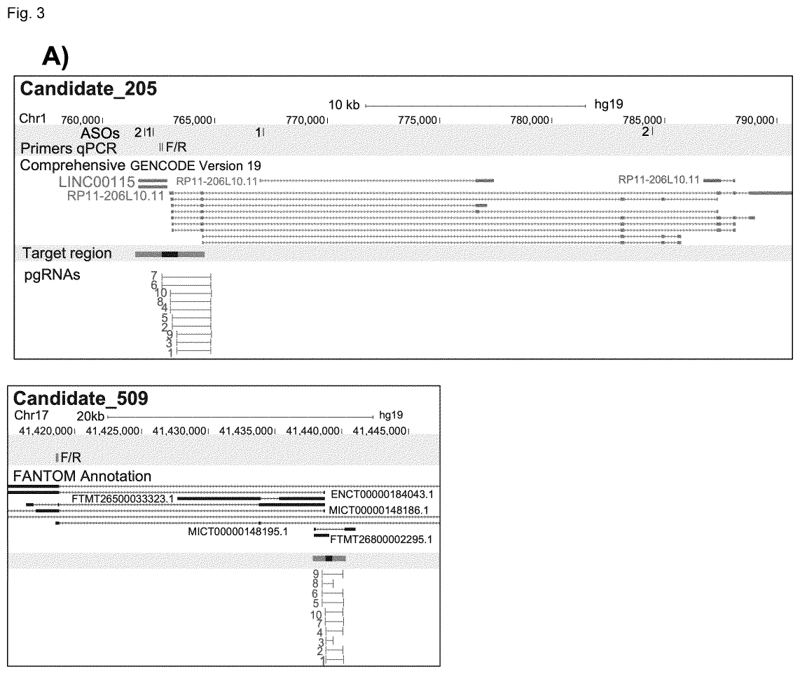

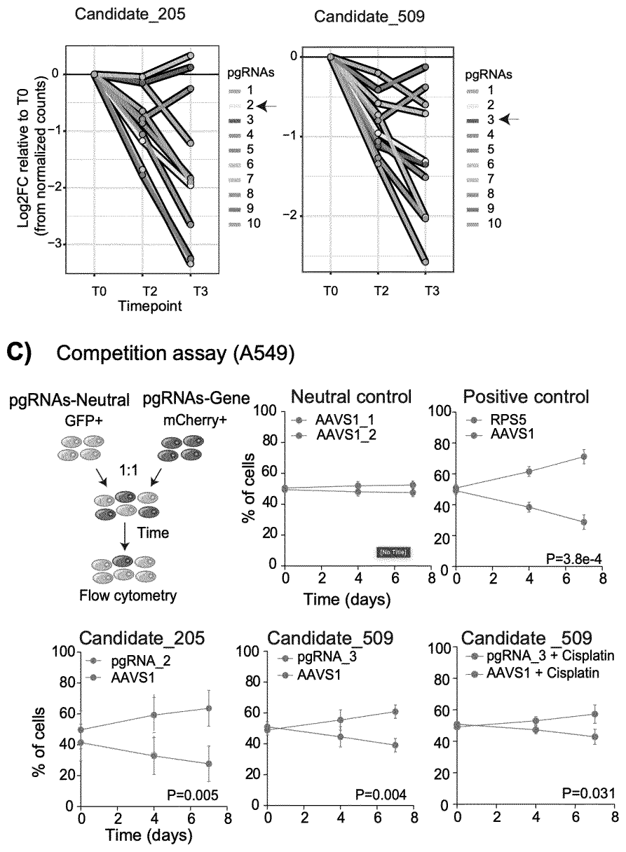

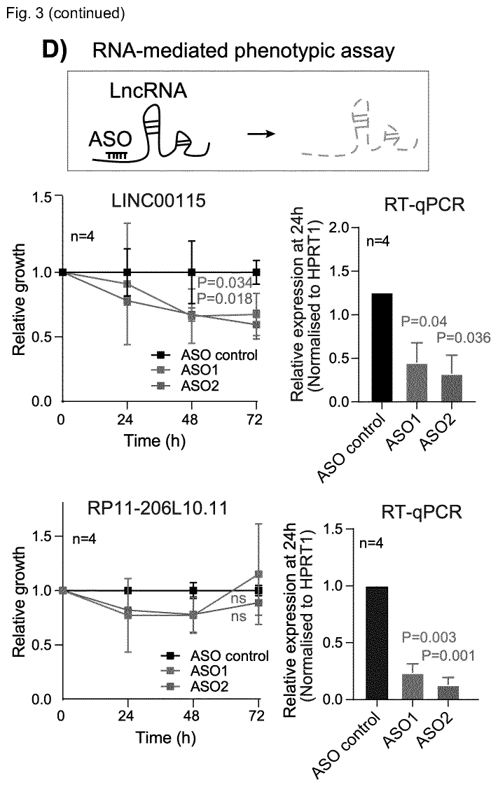

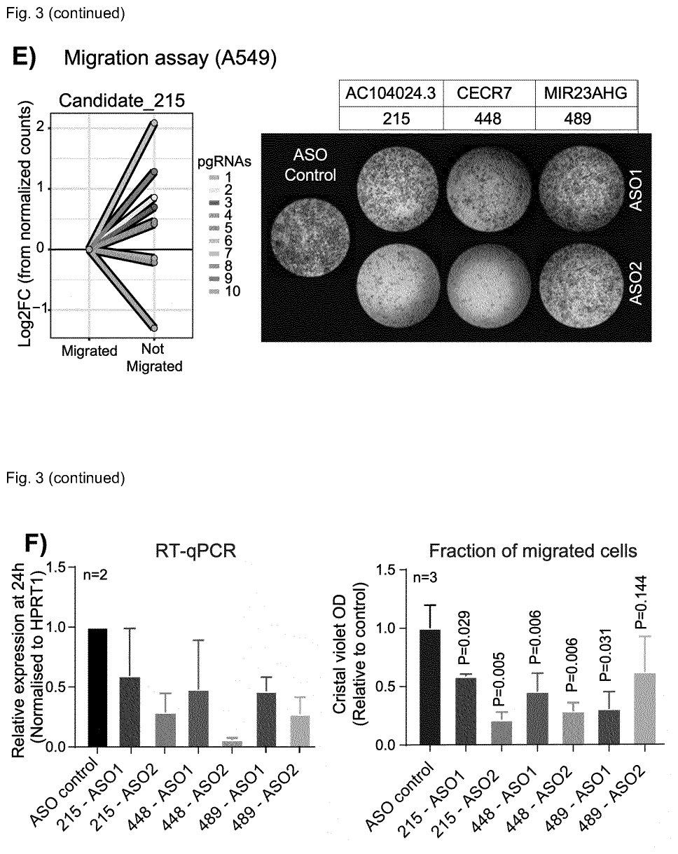

Candidate_205 overlaps the TSS of two bidirectional antisense lncRNA genes, LINC00115 and RP11-206L10, both from GENCODE annotation (FIG. 3A). Candidate_509 targets a TSS shared by several BIGTranscriptome lncRNAs (FIG. 3A). Supporting the importance of this locus, it contains two additional hits, Candidate_507 (LINC00910) and Candidate_508.

To validate the phenotypic effect of these deletions, the inventors re-cloned and tested individual depleted pgRNAs (FIG. 3B, arrows). Candidate_205 pgRNA efficiently deleted the targeted region, and while lack of PCR amplicons prevented direct testing of deletion by Candidate_509, it effectively decreased RNA levels. Both pgRNAs yielded strong effects on cell fitness: mCherry+ cells expressing pgRNAs were out-competed by control cells (GFP+, expressing pgRNA for AAVS1 locus), with an effect comparable to inactivation of the essential ribosomal gene, RPS5 (FIG. 3C). Similar results were found in a conventional cell growth assay. Furthermore, the pgRNA for Candidate_509 also sensitised cells to cisplatin, consistent with screen results (FIG. 3C, last panel).

It remained ambiguous which of the two divergent genes overlapping the Candidate_205 region is responsible for these effects. Furthermore, genomic deletion cannot distinguish between a DNA-dependent (for example, enhancer) or RNA-dependent mechanism (mature lncRNA, or its transcription). To address both questions, the inventors used two gene-specific ASOs to target each gene. This clearly implicated LINC00115, but not RP11-206L10, in driving cell proliferation via an RNA-dependent mechanism (Lai et al., 2020) (FIG. 3D). These results were corroborated in a three-dimensional (3D) spheroid model.

Similar high validation rates were observed for the migration screens. ASO-knockdown of three hits, Candidate_215 (AC104024.3), Candidate_448 (CECR7) and Candidate_489 (MIR23AHG) resulted in dramatic impairment of A549 migration (FIGS. 3E and 3F).

In summary, these findings support the ability of CRISPR-deletion screens to identify lncRNA genes that promote cancer hallmarks via RNA-dependent mechanisms.

### Multi-Hallmark Screen Integration for Target Discovery

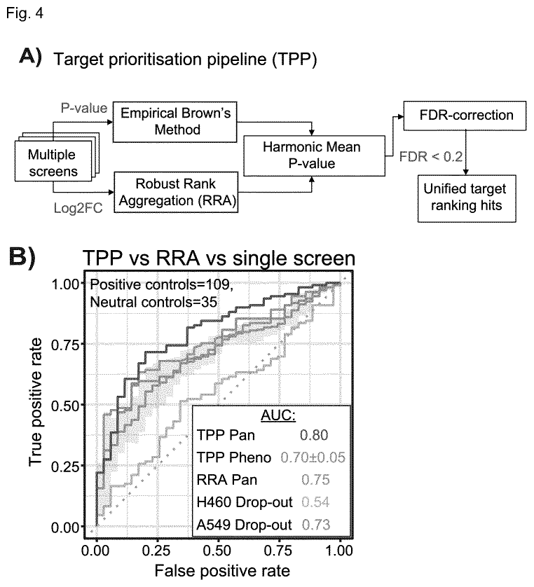

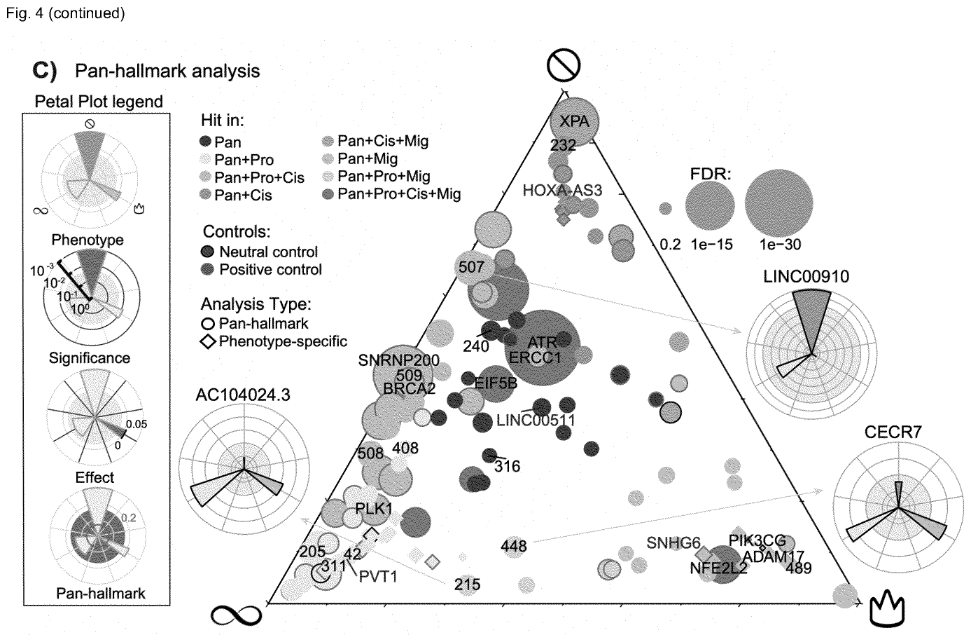

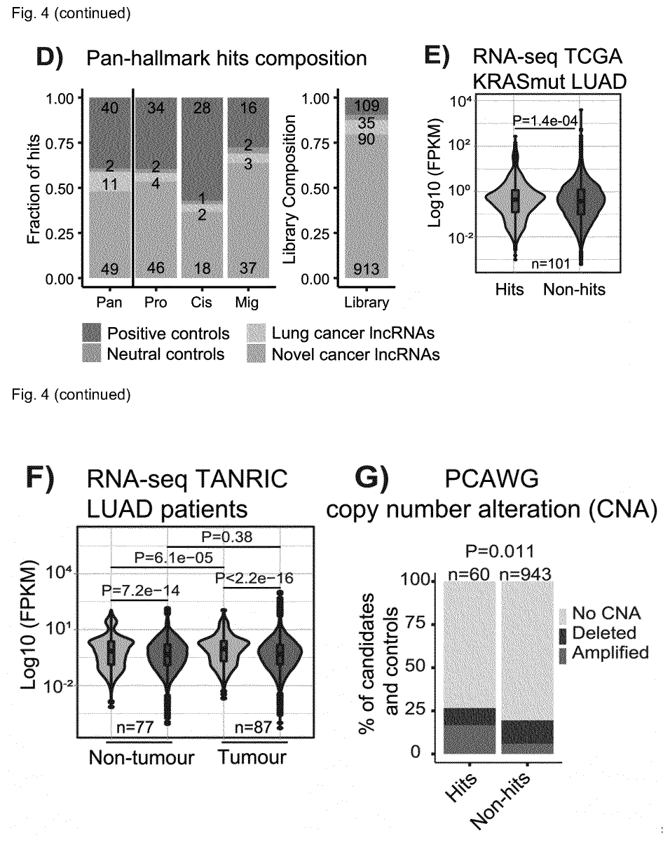

The inventors next sought to create an integrated, quantitative map of lncRNAs driving NSCLC hallmarks, by creating an integrative target prioritisation pipeline (TPP) (FIG. 4A). TPP enables integrating results from multiple CRISPR screens and while most previous studies prioritised hits by either effect size or statistical significance, TPP combines both to generate an integrated false discovery rate (FDR) for each gene. Its flexibility means that TPP can either be run for individual hallmarks (“hallmark-specific”), or on all screens for a “pan-hallmark” target ranking (see Methods). The pan-hallmark ranking outperforms other integrations and individual screens in correctly classifying positive and neutral controls (FIG. 4B).

These approaches provide the basis for a quantitative landscape of NSCLC lncRNAs: hallmark-specific values provide a signature for each lncRNA in three functional dimensions (displayed as petal plots), in the context of overall confidence defined by pan-hallmark ranking (FIG. 4C). The union of integrated and hallmark-specific screens yielded the 111 lncRNAs displayed in FIG. 4C. Pan-hallmark analysis identified altogether 60 lncRNAs (˜6% of those screened) (FDR<0.2), of which 49 were not previously linked to NSCLC (FIG. 4D). As expected, growth-promoting PCG controls and known lung cancer lncRNAs, but not neutral controls, are enriched amongst TPP hits. This is supported by more extensive Enrichment Score Analysis. Interestingly, hits are significantly enriched for disease-associated lncRNAs, and consistent with previous reports, identified cancer lncRNAs are more expressed in normal tissues but only marginally more evolutionarily conserved. Hallmark-specific integration yielded 96 hits (˜10% of those screened) in at least one hallmark, 14 in two hallmarks, and none in three.

Cancer-promoting lncRNAs are expected to be upregulated in tumours. Consistent with this, pan-hallmark hits are significantly higher expressed than non-hits in The Cancer Genome Atlas (TCGA) KRAS-mutated lung tumours (www.cancer.gov/tcga) (FIG. 4E), and are upregulated in tumour cells compared to adjacent normal tissue (FIG. 4F).

Finally, the clinical significance of screen hits is supported by the fact that they tend to be amplified, but not depleted, in tumour DNA from the Pan-Cancer Analysis of Whole Genomes Consortium (PCAWG) (FIG. 4G) and TCGA. Moreover, the inventors observed a significant correlation between the copy number status in A549 and H460 cells and the pan-hallmark analysis P-values (P=1.1e-06).

In summary, TPP integration of diverse screens yields accurate maps of functional lncRNAs that are enriched for meaningful clinical features.

RNA Therapeutics Targeting NSCLC lncRNAs

Multi-hallmark lncRNA maps are a resource for identifying therapeutic targets. ASOs are capable of effectively degrading lncRNAs both in vitro and in vivo, and have been approved for use in humans (Blokhin et al., 2018 Expert Opin. Drug Discov. 13, 837-849; Dhuri et al., 2020 J. Clin. Med. 9, 2004; Kaczmarek et al., 2017 Genome Med. 9, 1-16).

The inventors manually selected ten lncRNAs from top-ranked pan-hallmark and hallmark-specific hits, based on criteria of novelty and lack of protein-coding evidence, and henceforth referred to as “Tier 1”. Eight are annotated by GENCODE, and two by either FANTOM CAT or BIGTranscriptome. For each candidate, the inventors designed a series of ASOs and managed to identify at least two effective ASOs with >40% knockdown.

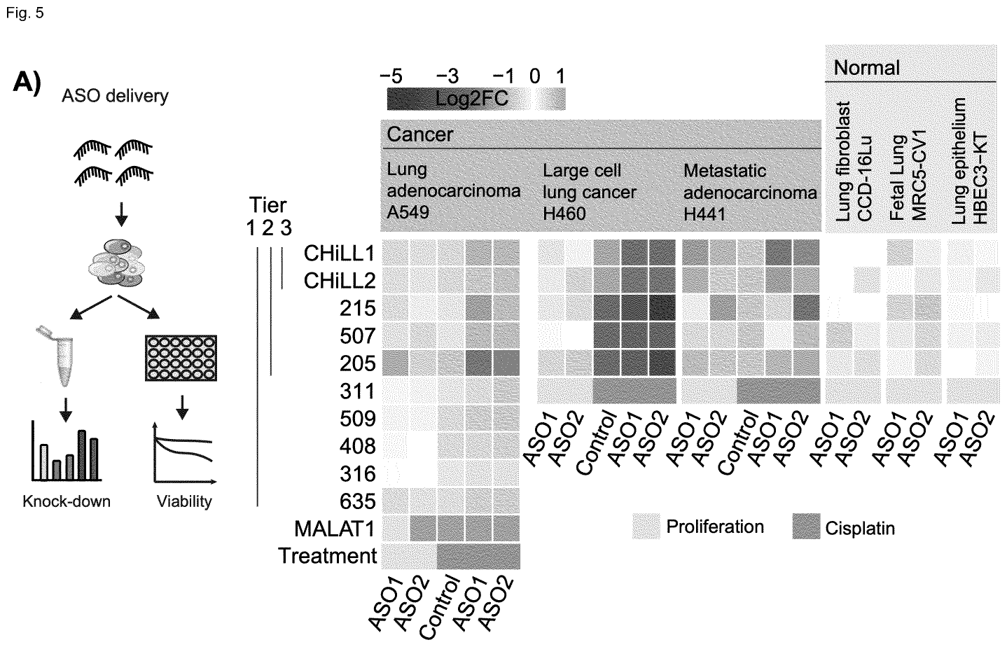

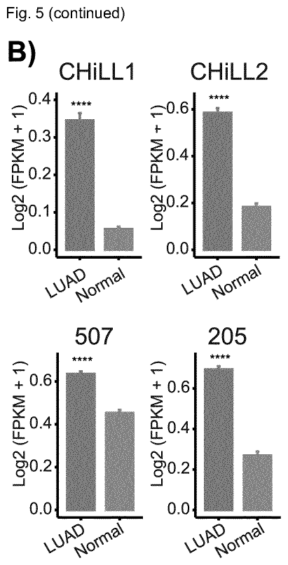

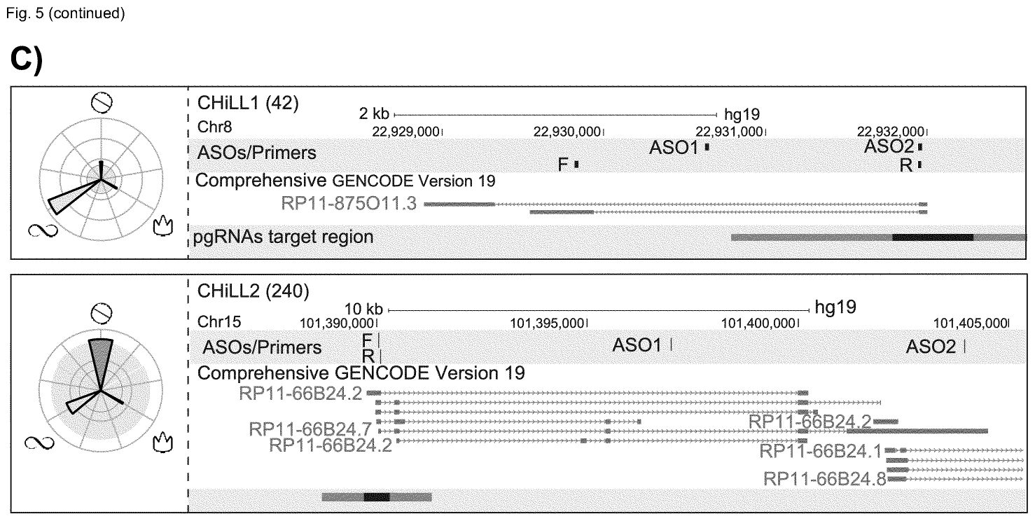

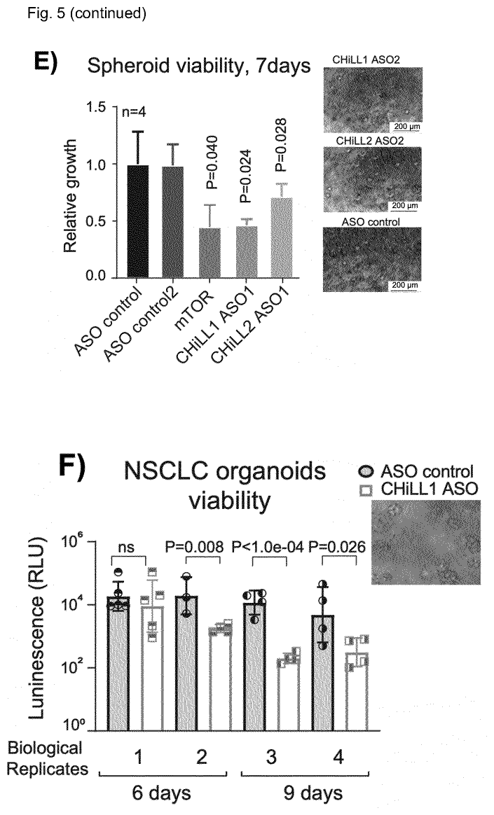

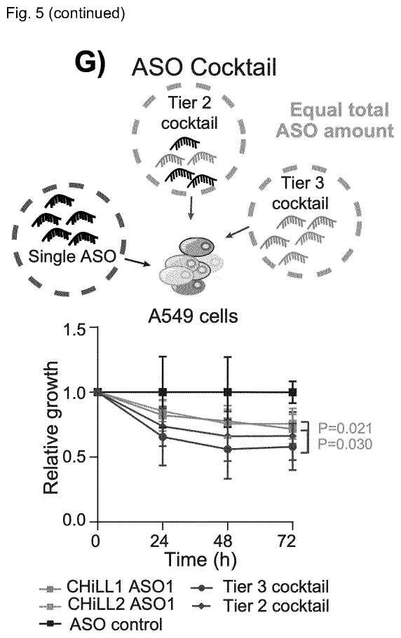

Next, the inventors tested ASOs' phenotypic effects, in terms of proliferation and cisplatin sensitivity (FIG. 5A). For five lncRNAs (Tier II), the inventors observed reproducible loss of cell proliferation with two distinct ASO sequences, indicating on-target effects. All are over-expressed in NSCLC tumours, despite this not being a selection criterion (FIG. 5B). To check how broadly applicable these effects are, the inventors re-tested the ASOs in two other KRAS-mutant NSCLC cell lines, H460 and H441 (derived from a pericardial effusion metastasis), and observed similar results (FIG. 5A). Consistent with their effects on cisplatin sensitivity, the inventors observed upregulation of Tier II genes' expression following cisplatin treatment.

It has been proposed that targeting lncRNAs could cause lower side-effects in healthy tissue, due to their tumour-specific expression, although few studies have tested this. To investigate their toxicity in healthy tissues, the inventors evaluated Tier 2 ASOs' effects on a panel of non-transformed lung-derived cells: HBEC3-KT, MRC5-CV1 (both immortalised) and CCD-12Lu (primary). These cells displayed diminished or absent response, particularly for the first two candidate lncRNAs (FIG. 5A). Consequently, the inventors narrowed the inventors' focus to these “Tier 3” lncRNAs: Candidate_42 (ENSG00000253616) and Candidate_240 (ENSG00000272808), henceforth renamed Cancer Hallmark in Lung LncRNA (CHiLL) 1 and 2, respectively. Both are annotated in GENCODE and have low protein-coding potential (FIG. 5C). Replicated experiments confirmed the knockdown efficiency and phenotypic impacts of both ASOs for each gene (FIG. 5D). ASOs displayed toxicity in additional KRAS-mutant and EGFR-mutant cell lines, suggesting subtype-independent activity.

CHiLL1 has, to the inventors' knowledge, never previously been implicated in cancer. It is located on Chr8 and consists of two annotated isoforms, sharing the first exon (FIG. 5C, upper panel), with expression restricted to lung and a handful of other organs. It is localized upstream and on the same strand of the protein-coding gene TNFRSF10B, previously associated with NSCLC, although the inventors find no evidence for read through transcription between the two loci. Its sequence lacks obvious functional elements. Supporting its relevance, high expression of CHiLL1 correlates with poor overall survival.

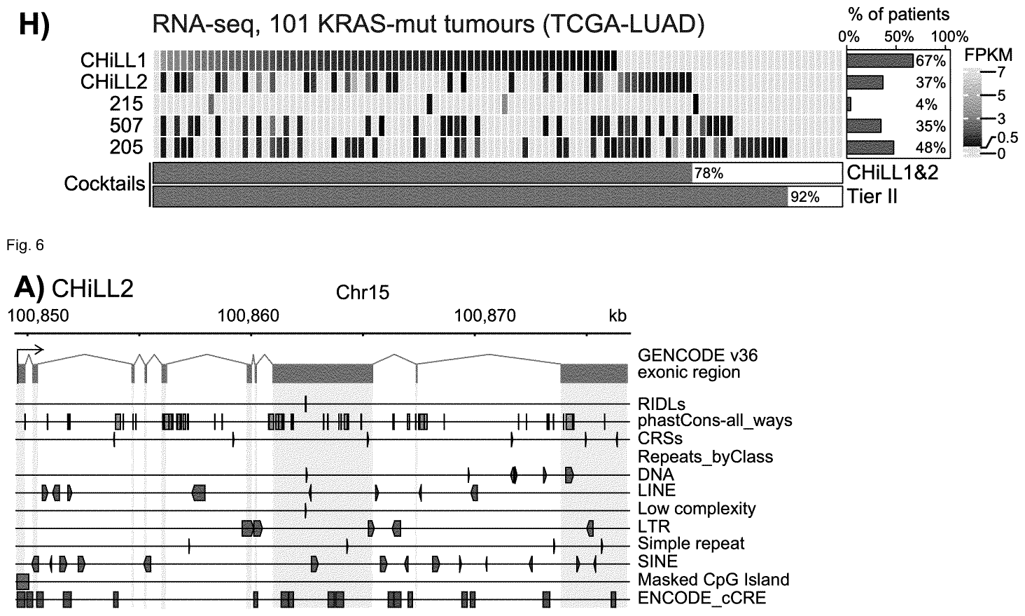

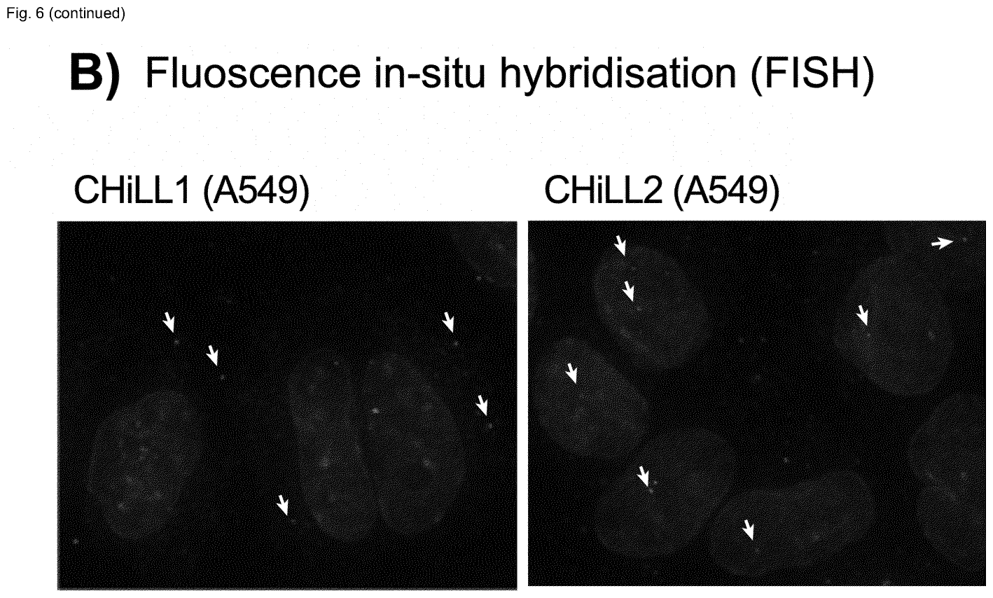

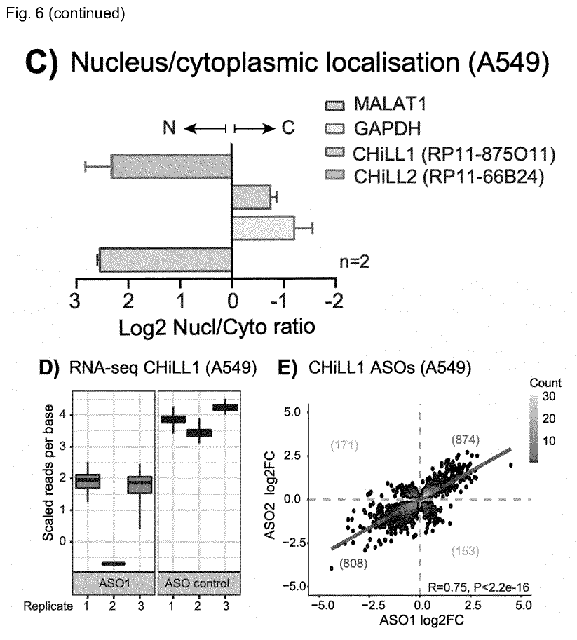

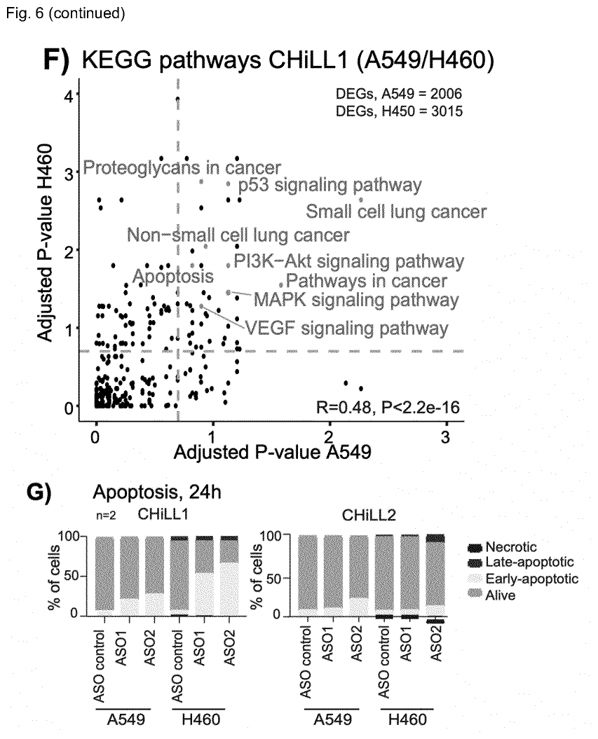

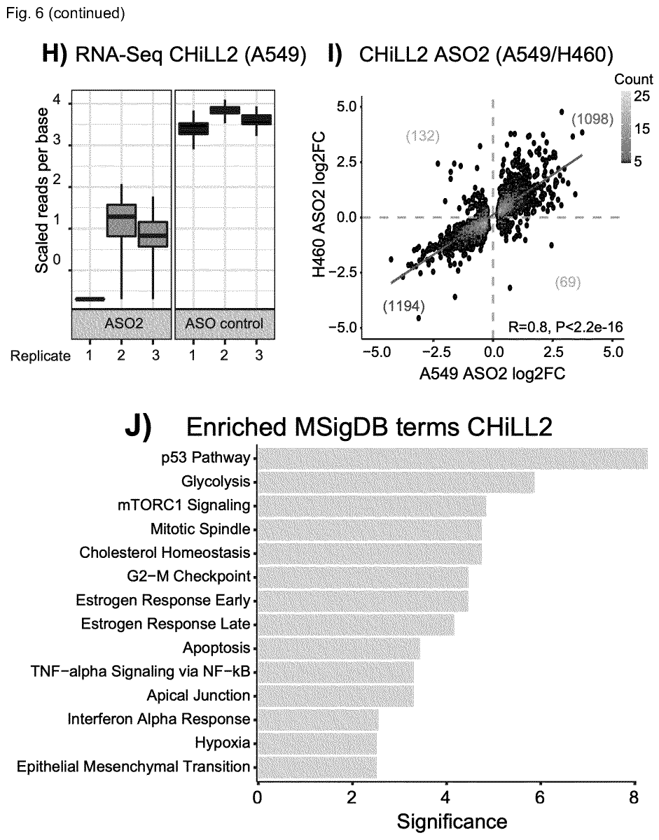

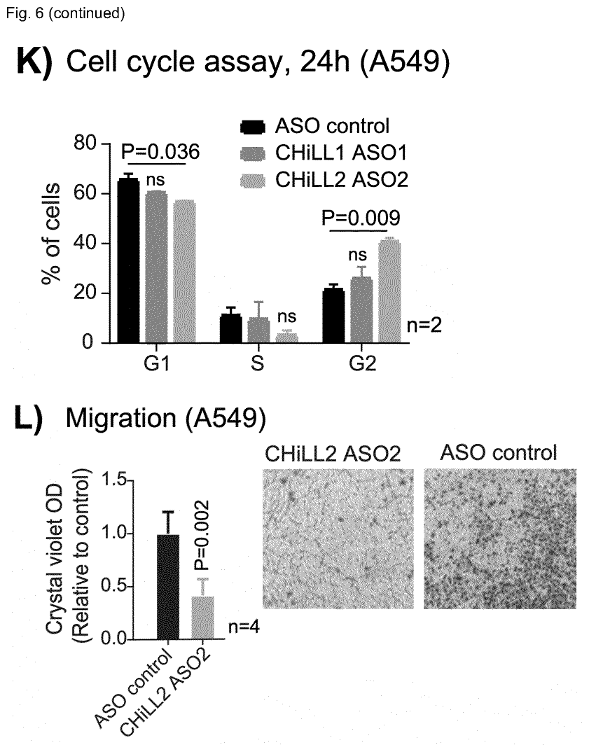

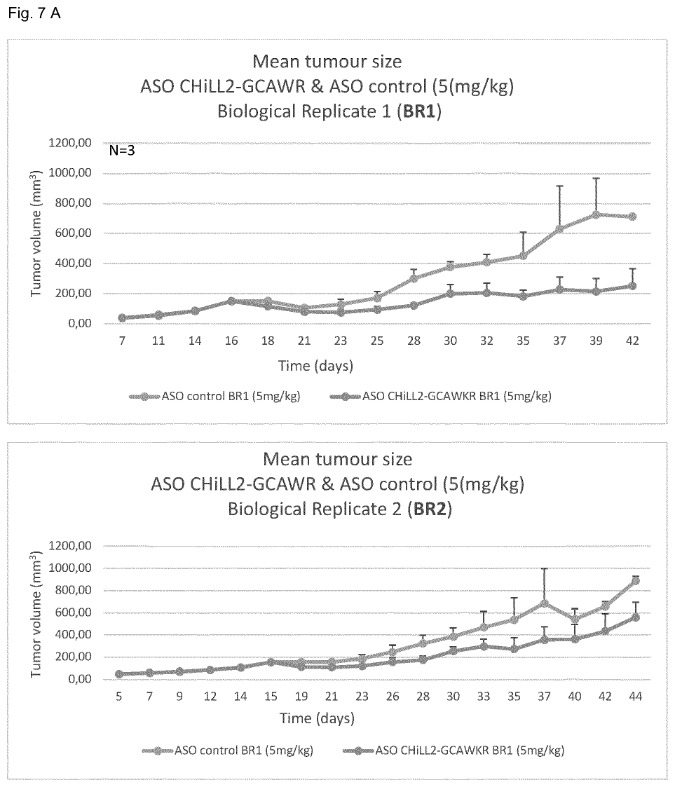

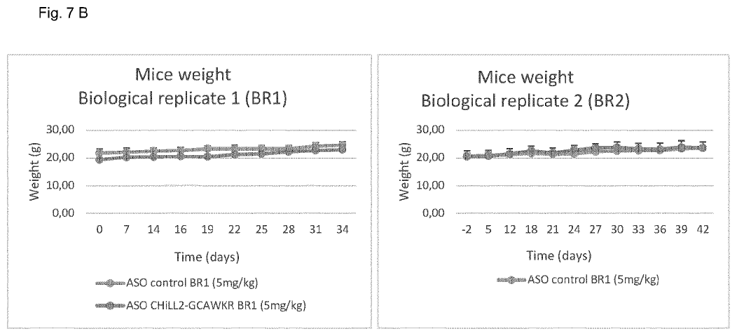

CHiLL2 (Chr15) comprises four isoforms sharing a common TSS (FIG. 5C, lower panel). It is associated with poor prognosis in colon cancer (Ruan et al., 2019 Oncol. Lett. 18, 1107-1116), and during preparation of this specification, was reported to be an oncogene in gastric cancer (Ma et al., 2018 Mol. Ther. 26, 2658-2668). It has a likely orthologue in mouse, the uncharacterised Gm44753. In contrast to CHiLL1, CHiLL2 exons contain numerous conserved sequences and structures (FIG. 6A), and its locus is frequently amplified in cancer genomes. In TCGA samples, CHiLL2 expression is upregulated in the proximal inflammatory (PI) tumour subtype, which is associated with poorer prognosis (PI vs. PP-P=4e-05; PI vs. TRU-P=0.008).

Three-dimensional (3D) in vitro models represent a more faithful tumour model compared to monolayer cultures. The inventors delivered CHILL1&2 ASOs to spheroid cultures of H441 cells, and observed a reduction in viability approaching that of the positive control, mTOR (FIG. 5E).

Organoids derived from patient-derived xenografts (PDX) recapitulate the therapy response of individual patients. Delivery of CHiLL1 ASOs resulted in significant reduction in cell viability of the KRAS-mutant human NSCLC organoid BE874 (FIG. 5F).

The above experiments demonstrate the effectiveness of inhibiting individual lncRNAs. The inventors were curious whether simultaneous targeting of two or more distinct vulnerabilities, via “cocktails” of ASOs, might offer synergistic benefits. Indeed, a 50:50 cocktail of CHiLL1/CHiLL2 ASOs (Tier 3 cocktail) displayed a greater effect on cell viability, compared to an equal dose of either ASO alone (FIG. 5G). A five-ASO cocktail targeting all Tier 2 lncRNAs yielded a similar benefit (FIG. 5G). Interestingly, cocktails resulted in no additional toxicity for non-cancerous cells.

Finally, the inventors asked what fraction of patients might benefit from ASO treatment. Using RNA-sequencing (RNA-seq) data from TCGA, the inventors estimate that 78% express at least one of CHiLL1 and CHiLL2 and might be treated by the Tier 3 cocktail, rising to 92% for Tier 2.

Together these results demonstrate that Tier 3 lncRNA can be targeted by potent and low-toxicity ASOs, and that combination therapy may yield additional benefits for a given dose in about 80% of KRAS-mutated patients (FIG. 5H).

CHiLL1&2 ASOs have Distinct Modes of Action

The inventors next investigated the modes of action linking CHiLL1&2 to NSCLC hallmarks. Subcellular localisation yields important mechanistic clues for lncRNAs. Surprisingly, despite their similar oncogenic roles, fluorescence in situ hybridisation (FISH) revealed contrasting localisation patterns: CHiLL1 is located principally in the cytoplasm, and CHiLL2 in the nucleus (FIG. 6B). Specificity was validated by knockdown and results were further corroborated by cell fractionation (FIG. 6C).

The inventors used RNA-seq to quantify the transcriptome of A549 cells treated with non-targeting and CHiLL1 ASOs. CHiLL1 expression in control cells was 8.31 TPM (transcripts per million), equivalent to ˜4 molecules per cell and consistent with FISH. RNA-seq confirmed knockdown efficiency by both ASOs (FIG. 6D), and resulting transcriptome changes were highly correlated, indicating that the majority of effects arise via on-target perturbation of CHiLL1 (FIG. 6E). Similar correlation was observed in H460 cells (not shown). The generality of CHiLL1-dependent expression changes was further confirmed by correlated expression changes between A549 and H460 (not shown).

To gain higher-level biological insights, the inventors explored perturbed genes using enrichment analysis. Defining high-confidence target gene subsets from the intersection of both ASOs, the inventors identified enriched KEGG terms (Kanehisa et al., 2017 Nucleic Acids Res. 45, D353-D361) (FIG. 6F). These underscored disease relevance (e.g. “Non-small cell lung cancer”), and also implicated potential mechanistic pathways (MAPK, P13K-Akt), and the high degree of concordance between the two cell backgrounds again supported the generality of CHiLL1 effects across KRAS-mutant NSCLC cells.

Similar analysis of the Molecular Signatures Database (MSigDB) implicated p53 and mTORC1 signalling. Numerous transcription factor binding sites (TFBS) are enriched in changing genes, including those recognised by known cancer regulators, such as ZBTB7A (in both A549 and H460).

Amongst the cell-type independent enriched KEGG pathways was “Apoptosis” (FIG. 6F). The inventors tested the notion that CHiLL1 knockdown acts via programmed cell death: Annexin V staining indicated that knockdown of CHiLL1, but not CHiLL2, results in a significant increase of early apoptotic cells (FIG. 6G).

Turning to CHiLL2 (mean expression 2.5 TPM, ˜1 copies per cell), the inventors observed effective knockdown and cell-type-independent effects using a single ASO (FIGS. 6H, 6I). Gene enrichment analysis revealed partially overlapping terms with CHiLL1 (including p53 pathway, cholesterol homeostasis and epithelial to mesenchymal transition). Amongst the CHiLL2-specific terms, the inventors noticed several related to cell cycle progression, including “G2-M checkpoint” (FIG. 6J). Indeed, knockdown of CHiLL2 led to an increase of cells in G2-phase (FIG. 6K). CHiLL2 targets are also enriched for migration genes, and knockdown led to impaired cell migratory capability (FIG. 6L).

Interestingly, the inventors notice that CHiLL2 knockdown resulted in a significant upregulation of CHiLL1, potentially explaining the additive effect on cell viability observed with the CHiLL1/CHiLL2 ASO cocktail (FIG. 5G).

Together, these data establish that CHiLL1&2 promote cancer hallmarks via widespread, non-overlapping downstream gene networks.

### DISCUSSION

The inventors have presented a powerful yet accessible target discovery pipeline for RNA therapeutics (RNATX), and used it to survey lncRNAs across hallmarks and mutationally-matched backgrounds in the most common KRAS-mutant subtype of non-small cell lung cancer. This reveals scores of mostly new therapeutic vulnerabilities, including two oncogenic lncRNAs, CHiLL1 & 2, whose suppression by ASOs reduced proliferation, chemoresistance and migration.

Pooled CRISPR screening is being rapidly adopted as the workhorse of functional genomics, thanks to its practicality and versatility. Screens can be performed in an average molecular biological laboratory without sophisticated robotics or arrayed libraries. Despite their practicality, they represent a powerful, independent window onto therapeutic vulnerabilities, independent of conventional parameters like survival, mutation, differential expression or evolutionary conservation. Nonetheless, key barriers to entry remain, including lack of screening libraries for lncRNAs. Targeting almost 1,000 carefully curated and disease-specific lncRNAs, libDECKO-NSCLC1 represents an important resource that is freely available to other researchers. This work also sets new technical standards for CRISPR screens. While previous studies have employed a single cell model, with a single hallmark (often proliferation) and in a single format, often drop-out, the inventors here took full advantage of the CRISPR's versatility to screen lncRNAs in multiple phenotypic dimensions and both positive and negative formats. The inventors solved the challenge of integrating diverse screen data, by developing the simple and robust TPP pipeline that balances effect size and significance.

The integrated dataset created here represents an unprecedented functional panorama of 998 lncRNAs driving phenotypic hallmarks in a single disease. This is the first attempt to functionally interrogate the entire expressed lncRNA landscape at hallmark resolution and in matched cellular backgrounds.

Using this dataset as a discovery resource, the inventors shortlisted twelve lncRNAs for ASO development, from which the inventors identified a pair of particularly promising oncogenic lncRNAs, CHiLL1 & 2. Their sensitivity to ASOs indicates that both act via an RNA transcript, or at least the production thereof. Both have profound effects on the cellular transcriptome, affecting hundreds of target genes that converge on many shared, oncogenic pathways. Interestingly, however, these effects are mediated by very different immediate molecular mechanisms, as evidenced by their distinct subcellular localisation, non-overlapping target genes, and the fact that mixing their ASOs yielded greater than additive effects.

CHiLL1 & 2 are promising therapeutic targets for several reasons. Firstly, replication experiments using different ASO sequences and different cell backgrounds confirmed that observed phenotypic and molecular effects occur on-target (via the intended lncRNA). Second, those phenotypic effects were diminished in non-transformed cells, pointing to reduced non-specific toxicity in vivo, although this will have to be addressed in future experiments. Third, they were effective in both monolayer and three-dimensional KRAS-mutant NSCLC backgrounds, in addition to several EGFR-mutant cell lines, raising hope for a more general efficacy. Future efforts will be required to further refine ASO sequences and chemistry, and to effectively deliver them in vivo.

ASO-based RNA therapeutics promise key advantages over present-day precision oncology. The first is drug design: small molecules are challenging to design in silico, creating the need for complex arrayed screening projects with large chemical libraries. In contrast, ASOs can be designed and synthesised by commercial vendors, rapidly and at low cost. A second advantage is target space: whereas typical tumours harbour on average ˜4 identifiable driver mutations (and often none at all), they express hundreds of cancer-promoting lncRNAs. The third benefit arises from lncRNAs' unique properties: tumour-specific activity and patient-specific expression. These raise hopes for reduced toxicity in healthy cells, allowing not only higher doses, but also combination therapies to suppress therapy resistance. Another, is the possibility of precision therapy tailored to each patient's tumour lncRNA profile. The inventors have found evidence supporting these latter points. Therapeutic target lncRNAs are differentially expressed within KRAS-mutant patients, and could be used in future to design tailored therapies based on ASO combinations. Furthermore, the inventors have shown how ASO cocktails boosted efficacy without increasing toxicity, compared to equal doses of single ASOs. The inventors' findings open the possibility of using either fixed cocktails or cocktails tailored specifically to a patient's tumour transcriptome, of potent, enduring, low-toxicity and personalised cancer treatment.

### In Vivo Experiments—CHiLL2

Animal experiments were approved by the local experimental animal committee of the Canton of Bern and performed according to Swiss laws for animal protection (license number: BE 12_21).

All procedures involving animals employed NSG mice, 7-week-old females, 25 g. The mice were injected with 1 million A549 cells (KRAS+ non-small cell lung cancer) subcutaneously. Once tumours reached 150 mm³, 5 mg kg⁻¹ of CHiLL2/GCAWKR-targeting ASO or control ASO (scramble ASO A from Qiagen) were injected next to the tumour every 3 days for up to 10 injections.

According to animal welfare guidelines, mice have to be killed when tumours reach a volume of 1,000 mm³ or when their body weight decreases more than 15% from the initial weight. Only one mice (from the control group) reached the limit at day 37 and had to be sacrificed. Notably, these mice did not suffer from any relevant adverse events or weight loss.

The results of this experiment are shown in FIG. 7

| | | | |
|---|---|---|---|
| CHILL2- | RP11-66B24 | ENSG00000272808 | CATAATCTGGGAACGA |
| GCAWKR | | | SEQ ID NO 15 |
| | | | GTGTGGTTGGAAGCTA |
| | | | SEQ ID NO 16 |

### Methods

### Cell Lines and Culture

HEK293T, A549, H460, H441, CCD-16Lu cell lines were a kind gift by the groups of Adrian Ochsenbein and Renwang Peng (University Hospital of Bern). MRC-5 cells were provided by the group of Ronald Dijkmanthe (Institute of Virology and Immunology, University of Bern). HBEC3-KT bronchial epithelial human cells were purchased from the American Type Culture Collection (ATCC; http://www.atcc.org). All the cell lines were authenticated using Short Tandem Repeat (STR) profiling (Microsynth Cell Line Typing) and tested negative for mycoplasma contamination.

A549 and HEK293T cells were maintained DMEM, MRC-5 in EMEM, NCI-H460, H441, and CCD-16Lu in RPMI-1640 medium, all supplemented with 10% Fetal Bovine Serum, 1% L-Glutamine, 1% Penicillin-Streptomycin. HBEC3-KT were maintained in Airway Epithelial Cell Basal Medium (ATCC®, cat. no. PCS-300-030) supplemented with Bronchial Epithelial Cell Growth Kit (ATCC®, cat. no. PCS-300-040).

All cells were passaged every 2-3 days and maintained at 37° C. in a humid atmosphere with 5% C02.

### Lentiviral Infection and Stable Cell Line Production

Lentivirus production was carried out by co-transfecting HEK293T cells with 12.5 μg of Cas9 plasmid with blasticidin resistance (Addgene, cat. no. 52962), 7.5 μg psPAX2 plasmid and 4 μg the packaging pVsVg plasmids, using Lipofectamine2000. 24 h before the transfection, 2.5e6 HEK293T cells were seeded in a 10 cm dish coated with Poly-L-Lysine (Sigma, cat. no. P4832) (diluted 1:5 in 1×PBS). The supernatant containing viral particles was harvested 24 h, 48 h and 72 h after transfection. Viral particles were then concentrated 100-fold by adding 1 volume of cold PEG-it Virus Precipitation Solution (BioCat, cat. no. LV810A-1-SBI) to every four volumes of supernatant. After 12 h at 4° C., the supernatant/PEG-it mixture was centrifuged at 1,500×g for 30 min at 4° C., resuspended in 1×PBS, and stored at −80° C. till use.

For the generation of stable Cas9-expressing cell lines, A549 and H460 were incubated for 24 h with culture medium containing concentrated viral preparation carrying pLentiCas9-T2A-BFP and 8 μg/ml Polybrene. Infected cells were selected for at least five days with blasticidin (8 μg/mL) and then were FACS-sorted two times, so as to have at least 60% BFP-positive cells.

### Design and Cloning of DECKO Plasmids and Lentiviral Production

For the design and cloning of DECKO plasmids, the inventors used their previously-described protocol (Aparicio-Prat et al., 2015 BMC Genomics 16, 846; Pulido-Quetglas et al., 2017 PLOS Comput. Biol. 13, e1005341) (http://crispeta.crg.eu/).

To produce lentivirus carrying the pDECKO plasmid, the inventors followed the same protocol. After infection with pDECKO plasmid-carrying viruses, cells were selected with puromycin (pg/mL) for at least three days.

### Library Design

The inventors downloaded GTF-format annotations from the following sources: i) GENCODE annotation release 19 (GRCh37) from gencodegenes.org; ii) BIGTranscriptome annotation (You et al., 2017 ibid.) from http://big.hanyang.ac.kr/CASOL/I; iii) FANTOM CAT (Hon et al., 2017 ibid.). The inventors also generated a novel transcriptome assembly of A549 RNA-seq (Davis et al., 2018 ibid; Dunham et al., 2012 ibid) using StringTie (Pertea et al., 2015 Nat. Biotechnol. 33, 290-295), version 1.3.

All lncRNAs were filtered thus: First, those with transcription start sites (TSS)<2 kb from any protein-coding gene exon were removed. Second, expression was calculated with RSEM v1.3 (Li and Dewey, 2011 BMC Bioinformatics 12, 323), and transcripts with FPKM <0.1 were removed. Remaining TSS within 300 bp were clustered into a single TSS. TSS were intersected with ENCODE evidence source specific to A549 cells: CAGE, DNAse I hypersensitivity sites and ChromHMM marks: Active TSS, Flanking TSS, Promoter Downstream TSS, Flanking TSS Downstream, Genic enhancer1, Genic enhancer2, Active Enhancer 1, Active Enhancer 2, Weak Enhancer and Bivalent-Poised TSS (Davis et al., 2018 ibid; Dunham et al., 2012 ibid; Ernst and Kellis, 2010 Nat. Biotechnol. 28, 817-825). Candidates were prioritized by the number of evidence sources.

The inventors designed neutral control pgRNAs in genomic regions not expected to affect cell phenotype. The inventors retrieved 10 regions in the AAVS1 gene loci from the publication of Zhu and colleagues (Zhu et al., 2016 Nat. Biotechnol. 34, 1279-1286). To this set the inventors added a set of 65 randomly selected intergenic regions (>10 kb distant from nearest gene annotation) and 25 intronic regions (for introns >5 kb in length). Moreover, 53 positive (promoting cell growth) and 50 negative (opposing cell growth) protein-coding gene (PCG) controls, with known roles promoting/opposing cancer cell growth and cisplatin resistance were added. These were manually selected from literature and retrieved from the paper of Zhu and colleagues ibid).

10 unique pgRNAs were generated for each candidate region with CRISPETa (Pulido-Quetglas et al., 2017 ibid) using the following parameters: -eu 0 -ed 0 -du 1000 -dd 1000 -si 0.2 -t 0,0,0,x,x-v 0.4 -c DECKO. For the candidates where <10 pgRNAs could be identified, the parameters were subsequently loosened until 10 were reached: in the second round one off-target with 3 mismatches was allowed; in the third round the designs region was repeatedly increased in size.

The final library design comprised 12,000 unique sequences of length 165/166 bp, with overhangs compatible with cloning into the pDECKO plasmid (Aparicio-Prat et al., 2015 ibid; Pulido-Quetglas et al., 2017 ibid).

### Library Cloning

Library was synthesized as single stranded oligonucleotides by Twist Bioscience (USA), and upon arrival resuspended in nuclease-free low Tris-EDTA (TE) buffer (10 mM Tris-HCl, pH 8.0 and 0.1 mM EDTA) to a final concentration of 10 ng/μl. This was PCR amplified using the Oligo-Fw: 5′-ATCTTGTGGAAAGGACGAAA-3′ (SEQ ID NO 013) and Oligo-Rev: GCCTTATTTTAACTTGCTATTTC (SEQ ID NO 014) with the following conditions: 95° C.×1 min; 10 cycles of (95° C. ×1 min, 53° C. ×20 sec, 72° C. ×1 min); 72° C. ×10 min. The amplification product was purified using the QIAquick PCR Purification Kit (Qiagen, cat. no. 28104) according to the manufacturer's instructions. The correct amplicon size was checked on a 2% agarose gel at 100V for 40 min.

The steps of cloning follow the low-throughput protocol described in Aparicio-Prat et al., 2015. In the first step, the pDECKO_mCherry plasmid was digested. The amplified library was inserted into the digested plasmid, using Gibson Assembly mix (obtained from ‘Biomolecular Screening & Protein Technologies’ Unit at CRG, Barcelona) at 50° C. for 1 h (200 ng of pDECKO_mCherry plasmid, 20 ng amplified library, H2O up to 10 μl, 10 μl of Gibson mix 10 μl). 1 μl of the Gibson reaction was delivered to 25 μl of electrocompetent Endura™ cells (Lucigen, cat. no. 60242-2) using Gene Pulser®/MicroPulser™ Electroporation Cuvettes, 0.1 cm gap (Biorad, cat. no. 16520891). The library coverage of 66.7× was estimated by counting the number of obtained bacterial colonies divided by the total number of different sequences in the designed library (12,000). The intermediate plasmid obtained in this step contains the pgRNA variable sequences, but still lacks the constant part of the first sgRNA and the H1 promoter (Aparicio-Prat et al., 2015 ibid).

In the second step of cloning, the intermediate plasmid was digested by Bmsbl enzyme (ThermoFisher, cat. no ER0451). After purification, the constant insert was assembled by ligation, by using PAGE purified and 5′ phosphorylated long oligos, as explained in Aparicio-Prat et al., 2015. Afterwards, 5 μl of the ligation product was transformed and used for the electroporation of electrocompetent Endura™ cells as described above. Clones were tested by colony PCR and by Sanger sequencing. The overall library quality was evaluated by NGS sequencing. Briefly, the plasmid containing the pgRNAs was amplified by PCR, purified using Agencourt AMPure XP beads (Beckman Coulter, cat. no. A63880), according to the manufacturer's protocol. The purified product was sequenced by Illumina at a depth of 20M PE125 reads. The reads were aligned to the pgRNAs library and the read distribution of each pgRNA was determined using the Ineq package in R (version 3.5.3) to calculate both the Lorenz-curve and Gini-coefficient.

### Lentiviral Titer Calculation and Lentiviral Infection

To achieve the desired multiplicity of infection (MOI) of 0.3-0.4, a titration experiment in A549 and H460 cells was performed. 2e6 cells were plated in each well of a 12-well plate and supplemented with 8 μg/ml polybrene. Each well was treated with virus ranging from 2.5 and 50 μl and transduced via spin-infection as previously described (Sanjana et al., 2014 Nat. Methods 11, 783-784). After centrifugation, the media was replaced with complete fresh media without polybrene and incubated overnight. The following day, cells were counted and each well was split in two equal aliquots, of which one was treated with 2 μg/ml puromycin. After 72 h, the MOI was calculated by dividing the number of surviving cells in the puromycin well, by the number in the puromycin-free well. The MOI of 0.3 was used for all screening experiments. For large-scale screens, 120M cells were seeded in 12-well plates with a density of 2M per well for spin-infection. The following day, cells were pooled together and fresh puromycin-containing (2 μg/ml) medium was added. Puromycin selection was maintained for six days until phenotypic screens began.

### CRISPR Screens

One week after infection (Timepoint 0 or TO), cells were counted and the reference sample was collected (TO, 16M cells corresponding to a library coverage >1,000×). For all screens, cells were cultured in 150 mm culture-treated dishes and passaged every 2-3 days.

Proliferation. Drop-out screens: at TO 16M of cells were plated and passaged so as to maintain a coverage >1,000× (defined as the number of cells divided by the number of unique library sequences). Cells were harvested at 14 and 21 days for gDNA extraction. CFSE screens: At T7, 16M cells were seeded and starved for 24 h with media lacking FBS. Then cells were stained using CellTrace™ CFSE Cell Proliferation Kit (ThermoFisher, cat. no. C34570) following the manufacturer's instructions. One aliquot of stained cells was immediately analyzed by flow cytometry, while the rest were plated with normal media. Five days later (T5), cells were sorted into two populations: 20% brightest (slow-growing) and 20% least bright (fast-growing). The two populations were plated separately and, five days later (T10) subjected to another round of staining and sorting.

Cisplatin screen. Optimal cisplatin working concentrations were established via dose response and cell doubling time. In the dose response, 3,000 A549 and H460 cells were plated in 96-well plates and treated with a range of cisplatin concentrations. After 72 h, CellTiter-Glo 2.0 (Promega, cat. no. G9242) was added to the media (1:1), and luminescence was recorded. For the cell doubling time, 1M cells were plated in 10 cm plates. Different cisplatin concentrations were added at indicated concentrations, and living cells counted every 2-3 days up to 14 days. Cisplatin survival screen: 48M and 96M cells were plated at TO and treated with 6.5 μM and 25 μM of Cisplatin for A549 cells and 2 μM and 10 μM for H460 cells, corresponding to IC30 and IC80, respectively. Cell pellets were collected after 14 and 21 days. The death screen was carried out as follows: 144M cells were seeded and treated with cisplatin at 2 μM and 1 μM (IC20) for A549 and H460, respectively. Every 24 h, for five days, floating (dead) cells were collected and pooled together for gDNA extraction.

Migration screen. To test the optimal conditions, the following set-up experiment was performed. 0.5 M A549 cells/well were seeded in 5 Boyden chambers (Corning, PC Membrane, 8.0 μm, 6.5 mm, cat. no. 3422-COR). Each migration assay was stopped at a different timepoint (ranging from 5 h up to 48 h). 48 h was selected as timepoint for the following experiment. At TO infected cells (˜16M) were divided and seeded in the upper part of 32 transwell inserts (0.5 M cells/transwell). The upper part of transwell inserts was filled with media lacking FBS, the lower part with media containing 10% FBS. After 48 h cells in the upper part of the chamber (impaired migration) and lower part (accelerated migration) (FIG. 2C) were trypsinized and plated separately for 48 h, after this time, cells were counted and collected for gDNA extraction. Control cells that did not undergo the migration assay were harvested at the same time as a reference population.

### Genomic DNA Preparation and Sequencing

Genomic DNA (gDNA) was isolated using the Blood & Cell Culture DNA Midi (5e6-3e07 cells) (Qiagen, cat. no. 13343), or Mini (<5e6 cells) Kits (Qiagen, cat. no. 13323) as per the manufacturer's instructions. The gDNA concentrations were quantified by Nanodrop.

For PCR amplification, gDNA was divided into 100 μl reactions such that each well had at most 4 μg of gDNA. Each well consisted of 66.5 μl gDNA plus water, 23.5 μl PCR master mix (20 μl Buffer 5×, 2 μl dNTPs 10 μM, 1.5 μl GoTaq; Promega, cat. no. M3001), and 5 μl of Forward universal primer, and 5 μl of a uniquely barcoded P7 primer (both stock at 10 μM concentration). PCR cycling conditions: an initial 2 min at 95° C.; followed by 30 s at 95° C., 40 s at 60° C., 1 min at 72° C., for 22 cycles; and a final 5 min extension at 72° C. PCR products were purified with Agencourt AMPure XP SPRI beads according to manufacturer's instructions (Beckman Coulter, cat. no. A63880). Purified PCR products were quantified using the Qubit™ dsDNA HS Assay Kit (ThermoFisher, cat. no. Q32854). Samples were sequenced on a HiSeq2000 (Illumina) with paired-end 150 bp reads at coverage of 40M reads/sample.

### Screen Hit Identification and Prioritisation

The raw sequencing reads from individual screens were analyzed by using CASPR (Bergadn-Pijuan et al., 2020 Bioinformatics 36, 1673-1680). After the mapping step, the obtained counts per million (cpm) for each pgRNA were filtered to remove sequences with 3>cpm>666. Low scoring guides were removed by GuideScan (Perez et al., 2017 Nat. Biotechnol. 35, 347-349), and a batch effect correction was applied using MageckFlute (Wang et al., 2019 Nat. Rev. Drug Discov. 19, 441-442). After all the corrections, the table count was provided to CASPR to calculate log 2-Fold Change and FDR corrected P-values at a target level.

To integrate multiple screens an integrative target prioritisation pipeline (TPP) was designed, applying two different approaches in parallel: the Robust Rank Aggregation (RRA) (Kolde et al., 2012 Bioinformatics 28, 573-580) to compute a ranking based on the effect size (CASPR log 2FC) across screens; and an empirical adaptation of Brown's method (EBM) (Poole et al., 2016 Bioinformatics, (Oxford University Press), pp. i430-i436) to combine the significance values (CASPR P-value) of each candidate across screens. The RRA-scores were converted to exact P-values using the rho-score correction from the same R package. Subsequently, the harmonic mean P-value (HMP) (Wilson, 2019 Proc. Natl. Acad. Sci. U.S.A 116,1195-1200) was calculated using the two significance scores from RRA and EBM. These P-values were corrected for multiple hypothesis testing using the Benjamini & Hochberg method, and a cutoff of FDR<0.2 was used to define hits.

Enrichment scores and nominal P-values (GSEA simulation, n=10,000) of positive and neutral control genes were used as indication for the quality of the ranking, as well as fraction of detected genes previously linked to lung cancer (Vancura et al., 2021 NAR Cancer 3). Positive and neutral control genes were also used as “true positives/false negatives” and “false positives/true negatives” respectively to calculate ROC curves and associated statistical metrics.

### Public RNA-Sequencing Data

101 KRAS-mutant LUAD RNA-seq samples were downloaded from TCGA (gdc.cancer.gov), applying the following filters: Adenocarcinoma—not treated—KRAS mutated, and the expression of target genes was estimated using HTSeq (Anders et al., 2015 Bioinformatics 31, 166-169). Another independent cohort of LUAD RNA-seq ex-vivo data, containing 87 tumour and 77 adjacent normal tissue samples, was obtained from the TANRIC (Li et al., 2015 Cancer Res. 75, 3728-3737; Seo et al., 2012 Genome Res. 22, 2109-2119).

PCR Amplification from Genomic DNA

gDNA was extracted with GeneJET Genomic DNA Purification Kit (ThermoFisher, cat. no. K0702) from pDECKO-transduced A549-Cas9-expressing cells. The PCR was done with primers flanking the deleted region as shown in FIG. 1F, using the Phusion™ High-Fidelity DNA Polymerase (2 U/μl) (ThermoFisher, cat. no. F-530S). The product was run on a 1% agarose gel.

### Competition Assay

A549 cells were infected with DECKO lentiviruses expressing fluorescent proteins. Viruses expressing control pgRNAs targeting AAVS1 also expressed GFP protein (pgRNAs-AASV1-GFP+), while the pgRNAs targeting candidate lncRNAs expressed mCherry. After infection, and seven days of puromycin (2 μg/ml) selection, GFP and mCherry cells were mixed 1:1 in a six-well plate (150,000 cells). Cell counts were analyzed by LSR II SORP instrument (BD Biosciences) and analyzed by FlowJo software (Treestar).

### Patient-Derived Xenograft Organoids

The KRAS-mutant patient-derived organoid BE874 was derived in the following way. Small pieces (˜1-2 cm³) of lung cancer tissue (provided by the Institute of Pathology, University of Bern) were taken from the surgically resected lung cancer specimen with patients' informed consent. Parts of the sample (pieces of around 5 mm) were separated and implanted subcutaneously into the flanks of 6 weeks old NOD.Cg-Prkdcscidll2rgtmlWjl/SzJ (NSG) mice (purchased from Charles River Laboratories) for cancer engraftment (Shultz et al., 2005 J. Immunol. 174, 6477-6489). After successful engraftment, tumour bearing mice were euthanized and tumours were resected. Single cells were isolated through mechanical and enzymatic tissue disruption for generation of BE874 organoids. Genotyping of BE874 organoids was performed at the Institute of Pathology, University of Bern, using KRAS targeted sanger sequencing. KRAS c.34G>T (p.Glyl2Cys) mutation was detected in both BE874 organoids and the corresponding primary cancer.

NSG mice were housed under specific pathogen-free conditions in isolated ventilated cages on a regular 12-hour/12-hour cycle of light and dark. Mice were fed ad libitum, and were regularly monitored for pathogens. Mouse experiments were licensed by the Canton of Bern and were performed in compliance with Swiss Federal legislation.

### Antisense Oligonucleotides

Locked nucleic acid ASOs were designed using the Qiagen custom LNA oligonucleotides designer (www.qiagen.com). Per each target, the inventors designed from 3 to 5 different ASOs. The day of the transfection, 300,000 cells were counted and plated on a 6-well plate. ASOs were transfected into the cells still in suspension, using Lipofectamine3000 (ThermoFisher, cat. no. L3000015) with final 25 nM in 2 ml media for A549, H460, NCI-H441, and MRC5-CV1 and 10 nM in 2 ml for HBEC3-KT and CCD-16LU, following the manufacturer's instructions.

For cocktail experiments the final concentration of the ASOs mix was kept at 25 nM. The media was refreshed 24 h post transfection and cells were harvested to check the efficiency of gene knockdown or sub-cultured for cell viability experiments. The inventors checked ASOs penetration in cells by means of the 5′-FAM-labeled control ASO A provided by Qiagen.

### 2D Cell Viability Assay

Cell viability assay was carried out in 2D cell lines by using CellTiter-Glo 2.0 (Promega, cat. no. G9242). The assays were performed according to the corresponding manufacturer's protocol. 24 h after the transfection, A549, H460, NCI-H441, H1975, H157, WVU-Ma-0005A, H820 and H1650 cells were harvested, counted and 3,500 cells/well were seeded in triplicate in 96 well plates. For Mrc5-SV1, HBEC3-KT and CCD-16LU 3,000, 3,500, and 1,000 cells/well were seeded, respectively. The number of viable cells was estimated after 24, 48, 72, 96 and/or 144 h. The day of the measurement, a mix of 1:1 media and CellTiter-Glo was added to the plates and the luminescence was recorded with Tecan Infinite® 200 Pro. Student's t test was used to evaluate significance (P<0.05).

### 3D Cell Viability Assay

NCI-H441 cells were detached, counted, and 200,000 cells were plated in 24 well plates. The ASO-Lipofectamine3000 mix was delivered to the cells in suspension as described above. After 24 hours, the cells are detached, counted and seeded onto 96-well Black/Clear Round Bottom Ultra-Low Attachment Surface Spheroid Microplate (Corning, cat. no. 4520) in 20 μl domes of Matrigel® Matrix GFR, LDEV-free (Corning, cat. no. 356231) and RPMI-1640 growth medium (1:1) with a density of 20,000 cells per dome. Matrigel containing the cells was allowed to solidify for an hour in the incubator at 37° C. before adding DMEM-F12 (Sigma, cat. no. D6421) media on top of the wells (40 μl and 80 μl for the wells intended to the first and second timepoint, respectively. The spheroids were allowed to grow in the incubator at 37° C. in a humid atmosphere with 5% CO2. After 4 h the number of viable cells in the 3D cell culture was recorded as time point 0 (TO), CellTiter-Glo® 3D Cell Viability Assay (Promega, cat. no. G9682) was added to the wells, following the manufacturer's instructions and the contents transferred into a Corning® 96-well Flat Clear Bottom White (Corning, cat. no. 3610) for the reading with the Tecan Infinite® 200 Pro. After one week the measurement was repeated.

BE874 organoids were generated and expanded using a special lung cancer organoid (LCO) medium.

BE874 organoids were transfected with ASOs as described for the NCI-H441. 24 h after transfection the cells were detached, counted and seeded onto Corning®96-well Flat Clear Bottom White (Corning, cat. no. 3610) in 20 μl domes of LCO growth medium and Matrigel (1:1) with a density of 20,000 cells per dome. The Matrigel-containing PDX-organoids was allowed to solidify for an hour in the incubator at 37° C. before adding 80 μl LCO growth media on top. The organoids were allowed to grow in the incubator at 37° C. in a humid atmosphere with 5% CO2. After 24 h, 100 μl of CellTiter-Glo® 3D Cell Viability Assay (Promega cat. no. G9682) were added to the wells intended for the TO and the luminescence was recorded with Tecan Infinite® 200 Pro. After three days the 80 μl of LCO media were added to the wells to keep them from drying out. After one week, the media was aspirated and replenished with fresh 80 μl, before proceeding with the measurement with CellTiter-Glo® 3D.

### Apoptosis Assay

Annexin V and viability dye were used to detect early apoptotic and dead cells, respectively. 24 h after the transfection, cells were counted and 150,000 cells were re-suspend in 100 μl of 1×PBS. The viability dye (ThermoFisher, cat. no. 35111) was added (1:5,000) in 100 μl of 1×PBS and cells incubated for 30 minutes at 4° C. Cells were then washed once with 1×PBS and re-suspend in 100 μl of Annexin buffer PH 7, added PE Annexin (1:200; ThermoFisher, cat. no. L34960) and incubated 30 minutes at 4° C. After a wash with 1×PBS, cells were resuspended in 300 μl of Annexin buffer and underwent the flow analysis by using the LSR Fortessa instrument (BD Biosciences). Unstained cells were used as control.

### Cell Cycle Assay

Cells were transfected with ASOs using Lipofectamine3000 according to the manufacturer's instructions. 24 h after, cells were harvested and fixed with 100 μl of BD Cytofix/Cytoperm Fixation (BD Biosciences, cat. no. 51-2090KZ) for 30 minutes at room temperature. The cells were then washed with 200 μl of 1× BD Perm/Wash (BD Biosciences, cat. no. 51-2091KE) and resuspended in 100 μl of 1×PBS. The K-i67 Antibody (ThermoFisher cat. no. 12-5698-82) was added (1:100) and incubated for 30 minutes at 4° C. Wash again with 1× BD perm/wash and stain with DAPI (Roche, cat. no. 10236276001) was added (1:10,000) in 100 μl of 1×PBS. Incubate 5′ at room temperature and wash with 1×PBS. Acquire the data with the Fortessa flow cytometer. Data analysis performed using FlowJo, and the different cell cycle phases were determined according to the Dean-Jett Fox (DJF) model.

### Low-Throughput Migration Assay

Migration assay was performed as previously described (Esposito et al., 2019 Cancer Res. 79, 2124-2135). 24 h after ASOs transfection, A549 cells were counted and seeded in the upper part of Boyden chambers with the density of 35,000 cells/transwell. The upper part of transwell inserts was filled with media without FBS, while the lower part with media supplemented with 10% FBS to induce the directional movement of cells. After 24 h the cells were washed three times with 1×PBS and stained using 300 μl of crystal violet 1% for 30 minutes. Three washes with 1×PBS followed. The cells in the upper part of the membrane were removed by using a cotton swab. The chambers are left to dry overnight. The day after, the crystal violet was solubilized in 1×PBS containing 1% SDS and the absorbance at 595 nm was recorded by using the Tecan Infinite® 200 Pro.

RNA Isolation and qRT-PCR

To purify total RNAs from cultured cells, a Quick-RNA™ kit from ZymoResearch was used according to the manufacturer's protocol. RNAs were reverse transcribed to produce cDNAs by using the GoScript™ Reverse Transcription System Kit (Promega, cat. no. A5003). The cDNAs were then used for qPCR to evaluate gene expression, using the GoTaq® qPCR Master Mix kit (Promega, cat. no. A6002). The expression of HPRT1 was used as an internal control for normalization.

### RNA-Sequencing and Analysis

24 h after ASO transfection, A549 and H460 cells were harvested and the total RNA was extracted as explained before and samples' quality was checked at Bioanalyzer. Libraries were prepared using the NEBNext® Ultra RNA Library Prep Kit and sequenced in paired-end 150 format to a depth of 30M reads/sample.

Transcript quantification was performed using Kallisto v0.46.0 (Bray et al., 2016 Nat. Biotechnol. 34, 525-527) against GENCODE v36 (Frankish et al., 2019 ibid). Gene level expression was inferred by aggregating the counts of the individual isoforms. Differential expression analysis was performed using Sleuth v0.30 (Pimentel et al., 2017). Genes with a q-value <0.2 were considered significant. For CHiLL1, the genes that were significantly up- and down-regulated with two different ASO were selected. For CHiLL2, the inventors selected the pool of common genes deregulated in A549 and H460.

### Fluorescent In-Situ Hybridization (FISH) and Cell Fractionation

FISH was performed on A549 cell lines, according to the Stellaris protocol (https://www.biosearchtech.com/support/resources/stellaris-protocols). For detection of CHiLL 1&2 at a single-cell level, pools of 25 and 48 FISH probes respectively were designed using the Stellaris probe designer software (www.biosearchtech.com). Cells were grown on round coverslip slides (ThermoFisher, 18 mm), fixed in 3.7% formaldehyde and permeabilized in ethanol 70% overnight. Hybridization was carried out overnight at 37° C. in hybridization buffer from Stellaris. Cells were counterstained with DAPI and visualized using the DeltaVision microscope.

Nuclear and cytoplasmic fractionation was carried out in A549 cells as described previously (Carlevaro-Fita et al., 2019a Genome Res. 29, 208-222.).

The comprehensive gene annotations for CHiLL1 (ENSG00000253616) and CHiLL2 (ENSG00000272808 and ENSG00000232386) loci were extracted from the GENCODE v36 GTF file (Frankish et al., 2019). Then, all the exons corresponding to each locus were collapsed into a meta-transcript and output as separate GTF files using BEDTools merge (Quinlan and Hall, 2010). Third, configuration files for each locus were prepared to draw the meta-transcript annotation alongside the genomic tracks using the program ezTracks (Guillen-Ramirez and Johnson, 2021).

### Copy Number Analysis of Pan-Hallmark CRISPR Candidates

The copy number status of A549 and in H460 cells was retrieved from the CCLE (https://portals.broadinstitute.org/ccle/data). Then, the inventors intersected the hg19 coordinates of the pgRNA with the hg19 coordinates of the CCLE copy number data.

TCGA-LUAD copy number data were downloaded as ‘log 2 ratio segment means’ using the R package TCGAbiolinks and converted to hg19 coordinates using liftOver. The values of each candidate were averaged across all TCGA-LUAD samples.

The pan-cancer recurrently amplified or deleted genomic regions were downloaded from the ICGC Data Portal (https://dcc.icgc.org/releases/PCAWG/consensus_cnv/GISTIC_analysis/all_lesions.conf_95.rmcn v.pt_170207.txt.gz). Then, the inventors searched for overlaps between each candidate and the recurrent copy number altered regions (“Wide Peak Limits”). The differences in the proportions of amplified, deleted, or non-copy number altered hits versus non-hits were tested using Fisher's exact tests.

### Sequences:

ENSG00000253616.1 (“CHiLL 1”; SEQ ID NO 001)
RP11-875011.3; Gene location: chr8:23,071,377-23,074,488 (-strand) (GRCh38 assembly coordinates) (see sequence protocol)
ENSG00000272808 (“CHiLL 2”; SEQ ID NO 002)
Gene location: chrl5:100,849,831-100,876,836 (+strand) (GRCh38 assembly coordinates) (see sequence protocol)

1. A method for treatment or inhibition of recurrence of non-small-cell lung cancer in a subject comprising, administering to a subject in need thereof a nucleic acid agent targeting and capable of downregulating or inhibiting a long non-coding RNA target selected from
a. ENSG00000253616 (CHiLL1; SEQ ID NO 001)
b. ENSG00000272808 (CHiLL2; SEQ ID NO 002).

2. The method of claim 1, wherein the target is ENSG00000253616 (SEQ ID NO 001).

3. The method of claim 1, wherein the target is ENSG00000272808 (SEQ ID NO 002).

4. The method of claim 1, wherein the agent is an antisense oligonucleotide.

5. The method of claim 1, wherein the nucleic acid agent comprises deoxyribonucleotides.

6. The method of claim 1, wherein the nucleic acid agent comprises phosphorothioate bonds connecting ribonucleoside units, dexoxyribonucleoside units and/or nucleoside analogue units,
particularly wherein the nucleic acid agent consists of dexoxyribonucleoside units and nucleoside analogue units connected by phosphorothioate bonds.

7. The method of claim 1, wherein the nucleic acid agent comprises LNA (2′O, 4′C methylene bridged RNA building blocks).

8. The method of claim 1, wherein
the target is ENSG00000253616 (SEQ ID NO 001) and wherein
a. the nucleic acid agent comprises or consists of the sequence CAGGAGAAAAGCACAC (SEQ ID NO 003) or
b. ATTCTGGGTCACTGCT (SEQ ID NO 004);
the target is ENSG00000272808 (SEQ ID NO 002) and wherein
c. the nucleic acid agent comprises or consists of the sequence CATAATCTGGGAACGA (SEQ ID NO 005) or
d. GTGTGGTTGGAAGCTA (SEQ ID NO 006).

9. A pharmaceutical composition comprising
a. a first nucleic acid agent targeting and capable of downregulating or inhibiting a long non-coding RNA target ENSG00000253616 (CHiLL1; SEQ ID NO 001) and
b. a second nucleic acid agent targeting and capable of downregulating or inhibiting a long non-coding RNA target ENSG00000272808 (CHiLL2; SEQ ID NO 002).

10. The pharmaceutical composition of claim 9, wherein the first nucleic acid agent and the second nucleic acid agent independently of one another:
a. are antisense oligonucleotides,
b. comprise deoxyribonucleotides,
c. comprise phosphorothioate bonds connecting ribonucleoside units, dexoxyribonucleoside units and/or nucleoside analogue units, particularly wherein the nucleic acid agent consists of dexoxyribonucleoside units and nucleoside analogue units connected by phosphorothioate bonds,
d. comprise LNA (2′O, 4′C methylene bridged RNA building blocks),
e. target ENSG00000253616 (SEQ ID NO 001) and wherein the nucleic acid agent comprises or consists of the sequence CAGGAGAAAAGCACAC (SEQ ID NO 003) or ATTCTGGGTCACTGCT (SEQ ID NO 004); or
f. target ENSG00000272808 (SEQ ID NO 002) and wherein the nucleic acid agent comprises or consists of the sequence CATAATCTGGGAACGA (SEQ ID NO 005) or GTGTGGTTGGAAGCTA (SEQ ID NO 006).

11. A method for treatment or prevention of recurrence of non-small-cell lung cancer, particularly for use in treatment or prevention of recurrence of lung adenocarcinoma, comprising administering to a subject in need thereof the pharmaceutical composition of claim 9.

12. The method of claim 1, wherein the nucleic acid agent is administered to a patient diagnosed with a tumour characterized by an activating KRAS mutation.

13. The method of claim 1, wherein the nucleic acid agent
a. targets CHiLL1 and the tumour is characterized by overexpression of CHiLL1 and/or
b. targets CHiLL2 and the tumour is characterized by overexpression of CHiLL2.

14. The method of claim 1, wherein the nucleic acid agent is administered to a patient diagnosed with a tumour characterized by resistance to chemotherapy,
particularly wherein the tumour is characterized by resistance to a drug comprising a platinum-containing complex,
more particularly wherein the tumour is characterized by resistance to a platinum-containing drug selected from carboplatin, satraplatin, cisplatin, dicycloplatin, nedaplatin, oxaliplatin, picoplatin, and/or triplatin tetranitrate.

15. The method of claim 1, wherein the nucleic acid agent is administered in combination with a drug comprising a platinum-containing complex,
particularly in combination with a platinum-containing drug selected from carboplatin, satraplatin, cisplatin, dicycloplatin, nedaplatin, oxaliplatin, picoplatin, and/or triplatin tetranitrate,
more particularly wherein the nucleic acid agent is administered in combination with a drug selected from carboplatin and cisplatin.
